(19) United States
(12) Patent Application Publication
(10) Pub. No.: US 2013/0331554 A1
(43) Pub. Date: Dec. 12, 2013

(54) ENRICHMENT AND CONCENTRATION OF SELECT PRODUCT ISOFORMS BY OVERLOADED BIND AND ELUTE CHROMATOGRAPHY

(71) Applicants: Asif Ladiwala, San Diego, CA (US); John Pieracci, Encinitas, CA (US)

(72) Inventors: Asif Ladiwala, San Diego, CA (US); John Pieracci, Encinitas, CA (US)

(73) Assignee: BIOGEN IDEC INC., Weston, MA (US)

(21) Appl. No.: 13/885,446

(22) PCT Filed: Nov. 15, 2011
(86) PCT No.: PCT/US11/60821

§ 371 Date: Aug. 30, 2013

Related U.S. Application Data
(60) Provisional application No. 61/413,908, filed on Nov. 15, 2010.

(51) Int. Cl. C07K 1/22

(52) U.S. Cl. CPC C07K 1/22
USPC 530/387.3

(57) ABSTRACT

Disclosed is a method for enhancing or increasing the concentration of biological product in a final mixture, wherein said biological product has one or more selected characteristics, wherein said method comprises: (a) allowing an initial mixture of biological products with and without said selected characteristics to contact a chromatography medium wherein the quantity of biological products in said initial mixture exceeds the binding capacity or the dynamic binding capacity of said chromatography medium; (b) allowing biological product not having said one or more selected characteristics to be separated by said chromatography medium; and (c) recovering a final mixture of biological products from said chromatography medium wherein said final mixture comprises an enhanced or increased concentration of biological product with one or more selected characteristics, compared to the concentration of biological product in said initial mixture.

### BACKGROUND OF THE INVENTION

1. Field of the Invention

The present invention relates to improved methods in the separation of biological molecules from complex mixtures. More specifically, the invention relates to improved methods for selectively increasing the homogeneity of biological molecules obtained from a heterogeneous mixture of molecules, particularly wherein such methods are used for large scale preparation and manufacturing processes.

2. Background Art

The present invention provides improved methods in the purification of biological molecules compared to other previously disclosed methods. Some examples of such other previously disclosed methods may be found in: Brown et al., WO 2006/099308 (PCT/US2006/008919), “A Method of Weak Partitioning Chromatography,” published Sep. 21, 2006; Pliura, et al., U.S. Pat. No. 5,439,591, “Displacement Chromatography Process,” issued Aug. 8, 1995; Pliura, et al., U.S. Pat. No. 5,545,328, “Purification of Hemoglobin by Displacement Chromatography,” issued Aug. 13, 1996; Kelley, et al., “Weak partitioning chromatography for anion exchange purification of monoclonal antibodies,” Biotechnol. Bioeng. 101(3):553-66 (2008); Brown, et al., “Overloading ion-exchange membranes as a purification step for monoclonal antibodies,” Biotechnol. Appl. Biochem. 56:59-70 (2010).

The present invention differs from previously described methods, at least in part, because previous methods describe a flow through mode of operation wherein impurities and undesirable product forms (or “isoforms”) bind to an adsorbent and the desired product is collected in the column flow through.

### BRIEF DESCRIPTIONS OF THE DRAWINGS

FIG. 1. Linear Gradient Run on SE HiCap.

FIG. 2. TSA Results for SE HiCap.

FIG. 3. Linear Gradient Run on TMAE HiCap.

FIG. 4. TSA Results for TMAE HiCap.

FIG. 5. Linear Gradient Run on Capto DEAE.

FIG. 6. TSA Results for Capto DEAE.

FIG. 7. Linear Gradient Run on cHT Type I.

FIG. 8. TSA Results for cHT Type I.

FIG. 9. TSA Results for Phenyl Sepharose 6FF (high sub).

FIG. 10. Overlaid Linear Gradient Elution Peaks at Different pH Conditions.

FIG. 11. Cumulative Recovery vs. Gradient Elution pH.

FIG. 12. Cumulative Sialylation vs. Gradient Elution pH.

FIG. 13. Cumulative HMW vs. Gradient Elution pH.

FIG. 14. Cumulative O-glycan Occupancy vs. Gradient Elution pH.

FIG. 15. Cumulative % Terminal GalNAc vs. Gradient Elution pH.

FIG. 16. Overlaid Breakthrough Curves at Different Load pH Conditions.

FIG. 17. Product Partitioning at pH 5.5.

FIG. 18. Product Partitioning at pH 6.5.

FIG. 19. Representative Chromatogram Obtained for Experiment Performed with 5× Diluted Biological product as TMAE HiCap Load.

FIG. 20. Normal Plot of Residuals for TSA.

FIG. 21. Plot of the Studentized Residuals vs. Predicted Values for TSA.

FIG. 22. Plot of the Studentized Residuals vs. Run Number for TSA.

FIG. 23. Plot of Predicted vs. Actual Values for TSA.

FIG. 24. BOX COX Plot for TSA.

FIG. 25. Contour Plot for TSA as a Function of Loading and Load pH.

FIG. 26. Contour Plot for TSA as a Function of Load pH and Load Conductivity.

FIG. 27. Determination of Operating Ranges for TMAE HiCap Process.

FIG. 28. Eluate HMW vs. Load Concentration.

FIG. 29. Multicomponent Isotherm for Monomer.

FIG. 30. Multicomponent Isotherm for HMW.

FIG. 31. Representative Chromatogram for the TMAE HiCap Overloaded Bind and Elute Process for the Enrichment of ExcR-Fc TSA.

### DESCRIPTION OF THE INVENTION

The present invention is an improvement over previously described methods, at least in part, because the present method utilizes an “overload bind and elute” mode of operation wherein a biological product is allowed to contact a chromatography medium (or other matrix) at a concentration or in an amount which exceeds the static or the dynamic binding capacity of the chromatography medium (or other matrix). This constitutes the “overload and bind” portion of the method of the present invention. During the overload and bind step, biological product having a selected characteristic (such as a high overall net-negative charge or a high sialic acid content) preferentially binds to the chromatography medium (or other matrix) while biological product (as well as other impurities) not having the selected characteristic, or having less of the selected characteristic (such as biological product having a lower overall net negative charge or a lower sialic acid content) is excluded or separated from the medium (or matrix). Subsequent to the overload and bind step, the bound target product is eluted (or otherwise dissociated or separated) from the chromatography medium (or other matrix) and recovered. Hence, the biological product mixture obtained has been enriched with a higher concentration of product having the selected (target) characteristic compared to the product mixture prior to application of the overload bind and elute purification step.

Methods of the present invention can be adapted and applied to the separation/purification of biological products based on any number of physical, biological, and/or chemical characteristics. For example, product isoforms may be selectively separated on the basis of charge and/or hydrophobicity by using appropriate adsorbents (such as, for example, strong or weak anion or cation exchange resins for charge based separations and hydrophobic adsorbents for separations based on hydrophobicity). Additionally, methods of the invention may be applied using mixed-mode chromatography (mixed-mode media) for separations based on two orthogonal product attributes (e.g., charge and hydrophobicity).

In one embodiment, product may be selectively enriched/separated, for example, based on peak product pI values wherein, for example, higher pI isoforms may be separated from lower pI, deamidated product isoforms on a cation exchange adsorbent.

In one embodiment, the present invention is useful for selectively enriching biological product isoforms (or “glycoforms”) wherein the selected or desired product characteristic is that of having increased or enhanced overall (total) levels of sialic acid content. In one embodiment, biological product with increased, total sialic acid content is obtained by overloading an anion exchange chromatography medium (e.g., TMAE HiCap) with a mixture of the biological product such that the concentration of total product exceeds the binding capacity, or the dynamic binding capacity, of the chromatography medium. Overloaded and undesired product (e.g., product with a lower sialic acid content, and other impurities) are allowed to flow-through the chromatography medium, then the selected, bound product (with high sialic acid content) is eluted (or otherwise separated or dissociated from the chromatography medium).

Embodiments of the invention are useful for obtaining highly homogeneous mixtures of a wide variety of biological products. Some examples of such biological products include, without limitation, proteins and protein fragments (i.e., full-length and partial length polypeptides/peptides), antibodies (immunoglobulins), heterologous fusion proteins, etc.

In one embodiment methods of the invention may be used for separation/purification of non-immunoglobulin proteins (or fragments thereof) fused with immunoglobulins (or domains, regions, of fragments thereof). In one embodiment, for example, methods of the invention are used for separation/purification of a fusion protein comprising an extracellular receptor ligand-binding domain linked (i.e., “fused”) with the Fc-region of an immunoglobulin (such as the Fc region of an IgG molecule). Antibody fusion proteins (e.g., Fc-fusion proteins) to which methods of the present invention may be applied are well known in the art, see for example, “Antibody Fusion Proteins,” edited by Steven M. Charnow & Avi Ashkenazi, Wiley-Liss, Inc., USA (1999) (ISBN 0-471-18358-X); and “Soluble Fc Fusion Proteins for Biomedical Research” by Meg. L. Flanagan et al. in “Methods in Molecular Biology: Monoclonal Antibodies: Methods and Protocols,” 378: 33-52 (2007), edited by M. Albitar, Humana Press Inc. USA (DOI: 10.1007/978-1-59745-323-3_{—}3). Some specific examples, without limitation, of Fc-fusion proteins to which methods of the invention may be applied include those such as described for example in Fung et al., U.S. Pat. No. 7,294,481 (issued Nov. 13, 2007); Drapeau et al., U.S. Pat. No. 7,300,773 (issued Nov. 27, 2007); and, Ryll et al., U.S. Pat. No. 6,528,286 (issued Mar. 4, 2003).

Embodiments of the invention include, without limitation, a method for enhancing or increasing the concentration of biological product in a final mixture, wherein said biological product has one or more selected characteristics, wherein said method comprises:

(a) allowing an initial mixture of biological products with and without said selected characteristics to contact a chromatography medium wherein the quantity of biological products in said initial mixture exceeds the binding capacity or the dynamic binding capacity of said chromatography medium;

(b) allowing biological product not having said one or more selected characteristics to dissociate or be separated from said chromatography medium; and

(c) recovering a final mixture of biological products from said chromatography medium wherein said final mixture comprises an enhanced or increased concentration of biological product with one or more selected characteristics, compared to the concentration of biological product in said initial mixture.

Embodiments of the invention include use of any known, or subsequently disclosed or developed, chromatography media or matrix. Examples, without limitation, of such media comprise: ion exchange media; anion exchange media; cation exchange media; hydroxyapatite media; hydrophobic interaction chromatography media; antibody-affinity media (e.g., Protein-A or variants thereof); immunoglobulin Fc-region affinity media (e.g., Fc-receptor affinity media); and, ligand-affinity media; receptor-affinity media; and mixed-mode media.

Embodiments of the invention include methods wherein the binding capacity or dynamic binding capacity of a chromatography medium is exceeded by: 10% or more; 20% or more; 30% or more; 40% or more; 50% or more; 100% or more; 200% or more; 500% or more; and 1000% or more.

Embodiments of the invention include methods wherein the binding capacity or dynamic binding capacity of said chromatography medium is exceeded by: 1.5-fold or more; 2-fold or more; 3-fold or more; 4-fold or more; 5-fold or more; 6-fold or more; 7-fold or more; 8-fold or more; 9-fold or more; 10-fold or more; 20-fold or more; 30-fold or more; 40-fold or more; 50-fold or more; 100-fold or more; and 500-fold or more.

Embodiments of the invention include methods wherein the amount of biological products recovered in the final mixture, compared to the amount of biological products in the initial mixture, is: about 10% to about 80% recovered; about 20% to about 60% recovered; about 30% to about 60% recovered; about 30% to about 50% recovered; about 35% to about 50% recovered; about 35% to about 45% recovered; about 40% to about 45% recovered; about 40% to about 50% recovered; about 45% to about 50% recovered; about 10% recovered; about 15% recovered; about 20% recovered; about 25% recovered; about 30% recovered; about 35% recovered; about 40% recovered; about 45% recovered; about 50% recovered; about 55% recovered; about 60% recovered; about 65% recovered; about 70% recovered; about 75% recovered; and about 80% recovered.

Embodiments of the invention include methods wherein the concentration of biological product with one or more selected characteristics is increased or enhanced, compared to the initial mixture of biological products, by: at least about 5%; at least about 10%; at least about 20%; at least about 30%; at least about 40%; at least about 50%; at least about 60%; at least about 70%; at least about 80%; and at least about 90%.

Embodiments of the invention include methods wherein the selected characteristic (or characteristics) comprises any one or more of: degree of net negative charge at a set pH value; degree of net positive charge at a set pH value; degree of hydrophobicity; degree of hydrophilicity; quantity and/or type of carbohydrate content; quantity and/or type of N-linked glycosylation content; quantity and/or type of O-linked glycosylation content; total sialic acid content.

Embodiments of the invention include methods wherein the biological product comprises: a protein; an antibody or fragment thereof; a polypeptide comprising an extracellular receptor ligand-binding domain; a receptor ligand; a heterologous fusion protein; a fusion protein comprising an immunoglobulin Fc-region; a fusion protein comprising an extracellular receptor ligand-binding domain and an immunoglobulin Fc-region.

Embodiments of the invention include methods for recovering a selected biological product at a manufacturing scale; including wherein the selected biological product is a therapeutically useful or beneficial compound.

### DEFINITIONS AND ABBREVIATIONS

Dynamic Binding Capacity (DBC):

The dynamic binding capacity of a chromatography media is the amount of target product the media will bind under actual flow conditions before significant breakthrough of unbound target product occurs. As this parameter reflects the impact of mass transfer limitations that may occur as flow rate is increased, it is much more useful in predicting real process performance than a simple determination of saturated or static binding capacity. See, Millipore Corp., Technical Brief, Lit. No. TB1175EN00 (2005). In general the lower the flow rates, the higher the dynamic capacity. Dynamic binding capacities are routinely determined by those of ordinary skill in the art. For example, DBC can be determined by loading a sample containing a known concentration of the target product, and monitoring for the product in the column flow-through while applying the sample. See e.g., Bioseparation and Bioprocessing, Vol. 1, Sect. 1.4.3 (edited by Ganapathy Subramanian, published by Wiley-VCH Verlag GmbH & Co. KGaA, Weinheim, Germany) 2^{nd} Ed.; 2007.

Binding Capacity/Static Binding Capacity:

Amount of target product the media will bind under static (non-flow through) conditions.

Overload/Overloading/Overloaded:

Exceeding the binding capacity or dynamic binding capacity of an adorbent or other affinity or product capturing medium or resin.
AEX: anion exchange chromatography
CEX: cation exchange chromatography
cHT: calcium hydroxyapatite
CV: column volume
DF: diafiltration
DoE: design of experiments
HIC: hydrophobic interaction chromatography
HMW: high molecular weight
PS: pilot scale
OD: optical density
TSA: total sialic acid
UF: ultrafiltration
WFI: water for injection

By applying knowledge within the skill of those in the art, embodiments of the invention may be modified and/or adapted for various applications, without undue experimentation, without departing from the general concept of the present invention. Therefore, such adaptations and modifications are intended to be within the meaning and range of equivalents of the disclosed embodiments, based on the teaching and guidance presented herein.

### EXAMPLES

### Example 1

### Summary

A chromatography process was developed for the isolation/purification/enrichment of biological products with enhanced or increased levels of sialylation compared to biological products with decreased or lower levels of sialylation present in the same initial mixture.

### 1 INTRODUCTION

The biological product utilized in the present example was an Fc-fusion protein comprising an extracellular receptor ligand-binding domain linked to the Fc domain of human a human immunoglobulin (hereinafter “ExcR-Fc”). The biological product has an apparent molecular weight of 130-150 kDa and a theoretical pI of 7.15. It was produced by recombinant DNA technology in a Chinese hamster ovary (CHO) cell mammalian expression system. The product has a complex carbohydrate profile with multiple potential N-glycans in the extracellular domain and Fc region. Additionally, the product has multiple potential O-glycans in the extracellular domain. These multiple potential sites for post-translational modification result in a heterogeneous product pool containing molecules with varying degrees of N- and O-glycan occupancy and varying levels of sialylation.

Highly sialylated forms of biological products can represent a highly desirable class of therapeutically advantageous protein isoforms. The present example describes development of a robust process capable of enriching the content of recovered/isolated, highly sialylated forms of biological product while maintaining acceptable product recovery and yield. In this particular example, a process was developed wherein TMAE HiCap chromatography was used to recover/isolate highly sialylated forms of an Fc-fusion protein.

Initially, a number of different adsorbents and chromatographic modes (i.e., CEX, AEX, HIC, and cHT) were screened for their ability to separate protein isoforms on the basis of the differences in the degree of sialylation. TMAE HiCap was selected because it provided improved resolution of product glycoforms and higher product yield as compared to the other tested adsorbents. Breakthrough experiments were performed on TMAE HiCap to measure the dynamic binding capacity of ExcR-Fc and the partitioning of glycoforms in the flow through mode. Based on these results, an “overloaded” bind and elute process was developed wherein more highly sialylated glycoforms were preferentially adsorbed and concentrated on the TMAE HiCap adsorbent, while the lower sialylated and non-sialylated glycoforms of ExcR-Fc flowed through the column. Subsequently, the column was eluted to recover the enriched higher sialylated glycoforms in the product pool.

Bind and elute experiments were performed on the TMAE HiCap column to optimize loading conditions to maximize enhancement of product sialylation and recovery. A Design of Experiments (DoE) approach was employed to evaluate process robustness and to identify the effect of the key process parameters on product quality and yield. Additionally, purification runs were performed under exemplary “worst-case” conditions to test the ability of the process to consistently meet desired product quality conditions.

### 2 MATERIALS

Chromatography Adsorbent
Fractogel® EMD TMAE HiCap (M)
Fractogel® EMD SE HiCap (M)
Bio-Rad cHT Type I
Phenyl Sepharose™ 6FF (high sub)

Columns
C0969: TMAE HiCap, 0.66 cm I.D.×15.7 cm bed height (1 CV=5.46 mL)
C1021: TMAE HiCap, 0.66 cm I.D.×14.5 cm bed height (1 CV=4.96 mL)
C1063: TMAE HiCap, 0.66 cm I.D.×15.3 cm bed height (1 CV=5.23 mL)
C1099: TMAE HiCap, 0.66 cm I.D.×15.3 cm bed height (1 CV=5.23 mL)
C1104: TMAE HiCap, 0.46 cm I.D.×10.0 cm bed height (1 CV=1.662 mL)
C1005: SE HiCap, 0.66 cm I.D.×16.2 cm bed height (1 CV=5.54 mL)
C0981: cHT Type I, 0.66 cm I.D.×16.1 cm bed height (1 CV=5.51 mL)
C1028: Phenyl Sepharose, 0.66 cm I.D.×16.1 cm bed height (1 CV=5.51 mL)

Equipment
ÄKTA Explorer 100 Chromatography Workstation (GE Healthcare, Piscataway, N.J.)

### 3 METHODS

The following sections provide a detailed description of experiments performed to develop and optimize a chromatography process for enrichment of the overall level of sialylation of ExcR-Fc product. Early development experiments (Sections 4.1-4.3) employed enzyme-based desialylation followed by fluorescent labeling of the released sialic acid and fluorimetric analysis for the quantitation of the TSA content (i.e., DMB method). However, this assay had low precision and sensitivity. Therefore, a new, more precise TSA assay that employed acid hydrolysis for desialylation followed by UV-HPLC analysis of the released sialic acids was developed and employed for subsequent development studies (Sections 4.4 and 4.5). In each analysis, a control sample was run as an internal assay control which allowed for the determination of relative TSA enhancement provided by the process steps and/or experimental conditions being evaluated in the study.

### 3.1 Adsorbent Screening

At the onset of development, linear gradient experiments were performed on cation exchange (SE HiCap), anion exchange (TMAE HiCap and Capto DEAE), and hydroxyapatite (cHT Type I) chromatography to identify chromatographic modes and adsorbents that provided good resolution for product glycoforms on the basis of the level of sialylation. These experiments used Phenyl Sepharose 6FF column eluate obtained from an early pilot-scale run (PS4) diafiltered into neutralized Prosep Ultra Plus elution buffer (i.e., 75 mM Acetate, pH 3.3 neutralized to pH 5.0 using 2.4 M Tris base).

Additionally, hydrophobic interaction chromatography on Phenyl Sepharose 6FF (high sub) was also evaluated for its ability to enrich the level of sialylation of ExcR-Fc. Hence, eluate samples from experiments performed during the development of a Phenyl Sepharose chromatography process were also assessed for Total Sialic Acid (TSA) analysis.

Once TMAE HiCap was selected as the adsorbent for further process development, additional linear gradient experiments were performed to evaluate the effect of pH (i.e., pH 5.5, 6.5, 7.5, and 8.5) on the resolution of various product glycoforms. These experiments used biological product from a pilot-scale run (PS10) diafiltered into 50 mM Acetate, pH 5.0 solution as the starting material. The following method was employed with the column effluent monitored at 280, 300 and 313 nm. Experiments were performed at a linear flow velocity of 150 cm/hr and all steps were operated in downflow.
1. The column was pre-equilibrated with 3 CVs of 50 mM Tris/3M NaCl, pH 8.0.
2. The column was equilibrated with 5 CVs of 50 mM Acetate, pH 5.5, 50 mM Bis Tris, pH 6.5, 50 mM Tris, pH 7.5, and 50 mM Tris, pH 8.5 for load pH levels of 5.5, 6.5, 7.5, and 8.5, respectively.
3. The column was loaded to 20 mg/mL with the starting material adjusted to the appropriate pH with 2.4 M Tris base.
4. The column was washed with 3 CVs of the equilibration solution.
5. The column was eluted with a linear salt gradient from 0-300 mM NaCl over 30 CV in the corresponding equilibration solution. At the end of the gradient, the % B was maintained at 100% for 2 CVs to wash the column with the high salt solution. The column eluate was collected in 2 CV fractions.
6. The column was stripped with 3 CVs of 50 mM Tris+3M NaCl, pH 8.0.
7. The column was cleaned with 3 CVs of 0.5M NaOH.
8. The column was regenerated with 3 CVs of 50 mM Acetate+1M NaCl, pH 2.5.
9. The column was stored with 3 CVs of 1% Benzyl alcohol+0.5M Acetic Acid+16 mM NaOH, pH 3.2

### 3.2 Breakthrough and Partitioning Experiments

Experiments were performed to evaluate the partitioning of glycoforms as well as the dynamic binding capacity (DBC) on TMAE HiCap as a function of load pH i.e., pH 5.5, 6.5, 7.5, and 8.5. These experiments employed biological product from a pilot-scale run (PS10) that was diafiltered into 50 mM Acetate, pH 5.0, diluted to approximately 5 mg/mL with DF buffer, and adjusted to the appropriate pH with 2.4 M Tris base. For each load pH condition, the TMAE HiCap column was loaded to 150 mg/mL and flow through fractions were collected in 10 mg/mL increments and analyzed for TSA levels. The following method was employed with the column effluent monitored at 280, 300 and 313 nm. Experiments were performed at a linear flow velocity of 150 cm/hr and all steps were operated in downflow.
1. The column was pre-equilibrated with 3 CVs of 50 mM Tris/3M NaCl, pH 8.0.
2. The column was equilibrated with 5 CVs of 50 mM Acetate, pH 5.5, 50 mM Bis Tris, pH 6.5, 50 mM Tris, pH 7.5, and 50 mM Tris, pH 8.5 for load pH levels of 5.5, 6.5, 7.5, and 8.5, respectively.
3. The UV monitor was auto-zeroed at the end of equilibration.
4. The column was loaded to 150 mg/mL with the load pool at the appropriate pH.
5. The column was washed with 3 CVs of the equilibration solution.
6. The column was eluted with 3 CVs of the corresponding equilibration solution containing 300 mM NaCl.
7. The column was stripped with 3 CVs of 50 mM Tris+3M NaCl, pH 8.0.
8. The column was cleaned with 3 CVs of 0.5M NaOH.
9. The column was regenerated with 3 CVs of 50 mM Acetate+1M NaCl, pH 2.5.
10. The column was stored with 3 CVs of 1% Benzyl alcohol+0.5M Acetic Acid+16 mM NaOH, pH 3.2

### 3.3 Preliminary Development

Based on the results of the partitioning experiments, preliminary bind and elute experiments were performed to determine the feasibility of using TMAE HiCap in overloaded mode for the purpose of enriching sialylated product glycoforms. These experiments employed PS10 biological product diafiltered into 50 mM Acetate, pH 5.0, diluted to approximately 5 mg/mL with DF buffer, and adjusted to pH 5.5 with 2.4 M Tris base. Column loadings of 100 and 150 mg/mL were examined and the corresponding eluate pools were analyzed by the TSA and SEC assays. The following method was employed with the column effluent monitored at 280, 300 and 313 nm. Experiments were performed at a linear flow velocity of 150 cm/hr and all steps were operated in downflow.
1. The column was pre-equilibrated with 3 CVs of 50 mM Tris/3M NaCl, pH 8.0.
2. The column was equilibrated with 5 CVs of 50 mM Acetate, pH 5.5.
3. The column was loaded to the target loading condition with the load pool at the appropriate pH.
4. The column was washed with 3 CVs of the equilibration solution.
5. The column was eluted with 8 CVs of 50 mM Acetate+300 mM NaCl, pH 5.5. Eluate pool collection was started at the start of the elution step and concluded when OD280 of ≦0.1 AU was reached.
6. The column was stripped with 3 CVs of 50 mM Tris+3M NaCl, pH 8.0.
7. The column was cleaned with 3 CVs of 0.5M NaOH.
8. The column was regenerated with 3 CVs of 50 mM Acetate+1M NaCl, pH 2.5.
9. The column was stored with 3 CVs of 1% Benzyl alcohol+0.5M Acetic Acid+16 mM NaOH, pH 3.2.

To evaluate the option of employing diluted biological product as the column load material without the need for a prior UF/DF step, overloaded bind and elute experiments were carried out with diluted biological product to evaluate the effect of load composition on column performance. To this end, PS10 biological product adjusted to pH 5.5 was diluted (i) 5× with WFI, (ii) 10× with WFI, and (iii) 5× with WFI followed by 2× dilution with 50 mM Acetate, pH 5.5. A bind and elute run at 100 mg/mL loading was performed for each load preparation using the method described above.

### 3.4 Design of Experiments (DoE) Study

A Design of Experiments (DoE) study was performed to identify suitable operating conditions for an overloaded bind and elute TMAE HiCap step to provide the desired level of enrichment of product sialylation while maintaining acceptable product yield. The key process parameters and their ranges investigated were: Column loading (90-160 mg/mL), load pH (5.2-5.8), and load conductivity (2.4-4.4 mS/cm). A central composite design comprising of 18 runs (including 4 center point experiments) was created using the Stat-Ease Design Expert v8.0 (Stat-Ease, Inc., Minneapolis, Minn.) software. For each DOE run, operating parameters were varied as per the DoE experimental design table (Table 1).

TABLE 1
ExcR-Fc TMAE HiCap Central Composite DoE Study Design

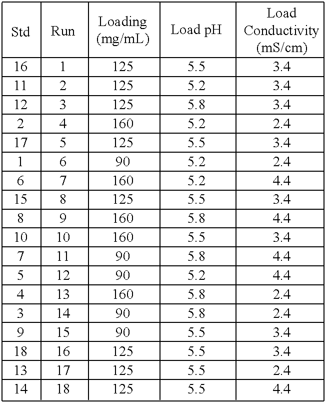

Note:
Shaded cells denote the center point runs.

Load material employed for the DoE experiments was generated from biological product obtained from representative a prototype run (PS 14) diafiltered into 50 mM Acetate, pH 5.5 and diluted to approximately 5 mg/mL with DF buffer. For each DoE run, the load was adjusted to the desired pH by titrating with 1M Bis Tris or 2N acetic acid, while the load conductivity was adjusted using WFI or a 500 mM acetate, pH 5.5 solution.

The two key process outputs—TSA and % HMW—were measured for each eluate pool and the software was employed to analyze the data and build models for each response. In addition, process recovery was also analyzed and modeled. The chromatography method described above was employed for these DoE experiments.

### 3.5 Additional Process Characterization

Additional single-point bind and elute experiments (Table 2) were performed on TMAE HiCap in order to:
1. Verify predictive ability of the DoE models and examine the effect of load lot variability on TMAE HiCap process performance (Runs 2, 5, 8);
2. Evaluate performance of TMAE HiCap adsorbent lot for use in large scale production (Runs 2, 5, 8);
3. Study effect of flow velocity on process performance (Runs 1-3 and 11-13);
4. Examine effect of load concentration on product quality and yield (Runs 4-6 and 7-9); and
5. Evaluate sialic acid (SA) enrichment and eluate HMW levels obtained under worst-case conditions for load TSA (Run 12) and load HMW (Run 10).

TABLE 2
Summary of Additional Process Characterization Experiments

| | | Column | | Load | Load | Load |
|---|---|---|---|---|---|---|
| | Load | Loading | Load | Cond. | Velocity | Conc. |
| Run # | Lot | (mg/mL) | pH | (mS/cm) | (cm/hr) | (mg/mL) |
| 1 | PS16 | 160 | 5.3 | 3.4 | 75 | ~5 |
| 2 | PS16 | 160 | 5.3 | 3.4 | 150 | ~5 |
| 3 | PS16 | 160 | 5.3 | 3.4 | 250 | ~5 |
| 4 | PS16 | 145 | 5.5 | 2.9 | 150 | ~3 |
| 5 | PS16 | 145 | 5.5 | 2.9 | 150 | ~5 |
| 6 | PS16 | 145 | 5.5 | 2.9 | 150 | ~7 |
| 7 | PS16 | 180 | 5.1 | 3.9 | 150 | ~3 |
| 8 | PS16 | 180 | 5.1 | 3.9 | 150 | ~5 |
| 9 | PS16 | 180 | 5.1 | 3.9 | 150 | ~7 |
| 10 | PS10 | 180 | 5.5 | 3.1 | 150 | ~5 |
| 11 | PS10 | 150 | 5.5 | 3.1 | 75 | ~5 |
| 12 | PS10 | 150 | 5.5 | 3.1 | 150 | ~5 |
| 13 | PS10 | 150 | 5.5 | 3.1 | 250 | ~5 |

PS10 and PS16 biological product separately diafiltered into 50 mM Acetate, pH 5.5 was employed for these experiments. For each run, the load was adjusted to the desired pH by titrating with 1M Bis Tris or 2N acetic acid, while the concentration and load conductivity were adjusted using WFI and/or 500 mM acetate, pH 5.5 solution. The following method was employed with the column effluent monitored at 280, 300 and 313 nm. Experiments were performed at a linear flow velocity of 150 cm/hr and all steps were operated in downflow.
1. The column was pre-equilibrated with 3 CVs of 50 mM Tris/3M NaCl, pH 8.0.
2. The column was equilibrated with 5 CVs of 50 mM Acetate, pH 5.5.
3. The column was loaded to the target loading condition with the load pool adjusted to the appropriate concentration, pH, and conductivity.
4. The column was washed with 3 CVs of the equilibration solution.
5. The column was eluted with 8 CVs of 50 mM Acetate+300 mM NaCl, pH 5.5. Eluate pool collection was started at the start of the elution step and concluded when OD280 of ≦0.1 AU was reached.
6. The column was stripped with 3 CVs of 50 mM Tris+3M NaCl, pH 8.0.
7. The column was cleaned with 3 CVs of 0.5M NaOH.
8. The column was regenerated with 3 CVs of 50 mM Acetate+1M NaCl, pH 2.5.
9. The column was stored with 3 CVs of 1% Benzyl alcohol+0.5M Acetic Acid+16 mM NaOH, pH 3.2.

Subsequently, additional single-point experiments were performed to further optimize the load concentration of the TMAE HiCap process step. Load concentrations between 2-30 mg/mL were evaluated. PS16 biological product diafiltered into 55 mM Acetate, pH 5.3 was employed as the starting material for these experiments. The diafiltered pool was adjusted to worst-case load pH and conductivity conditions for HMW (i.e., pH 5.1 and 3.9 mS/cm) and diluted to the appropriate target load concentration using a 55 mM Acetate adjusted to pH 5.1 and 3.9 mS/cm. Experiments were performed on a 1.66 mL (0.46×10 cm) screening column at a linear flow velocity of 100 cm/hr using the chromatography method described above. Note: The velocity for these runs was reduced in order to match the residence time on the 15 cm bed height development column.

### 4 RESULTS AND DISCUSSION

### 4.1 Adsorbent Screening

Linear gradient experiments were performed to screen various chromatographic modes and adsorbents in order to identify a suitable chromatography step for the enrichment of product sialylation. To this end, cation exchange (SE HiCap), anion exchange (TMAE HiCap and Capto DEAE), hydroxyapatite (cHT Type I), and HIC (Phenyl Sepharose 6FF) adsorbents were evaluated. Results of the TSA analysis performed for the eluate fractions for each run are shown below. These results clearly showed that TMAE HiCap provided the highest amount of SA enrichment at relatively higher process yield as compared to the other adsorbents evaluated in this study.

4.1.1 Fractogel SE HiCap

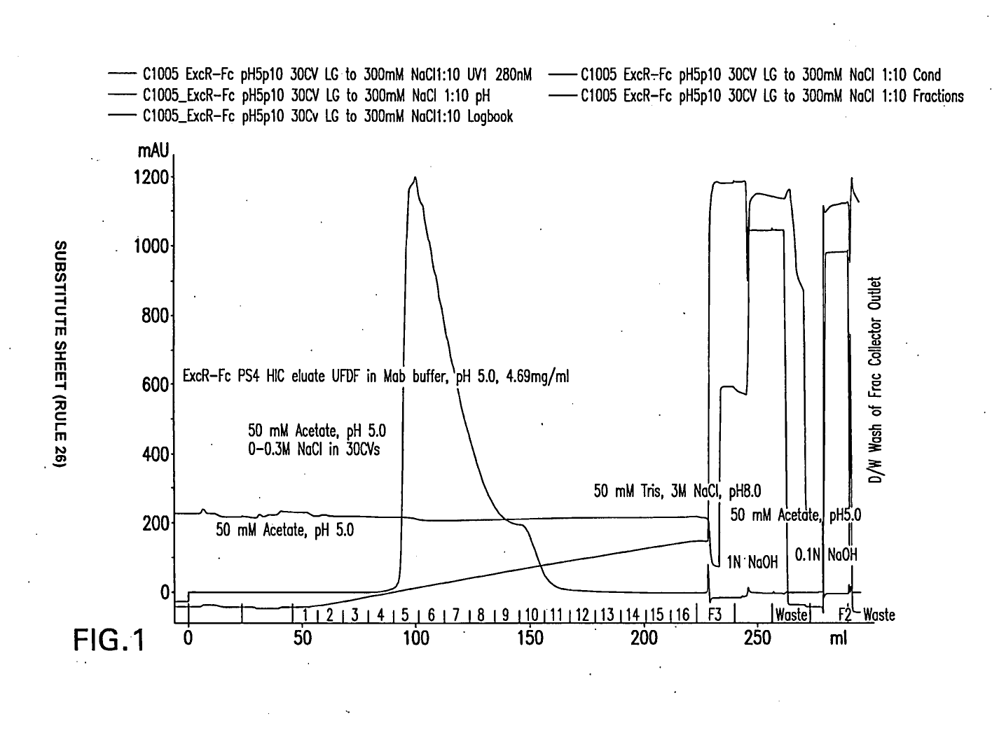

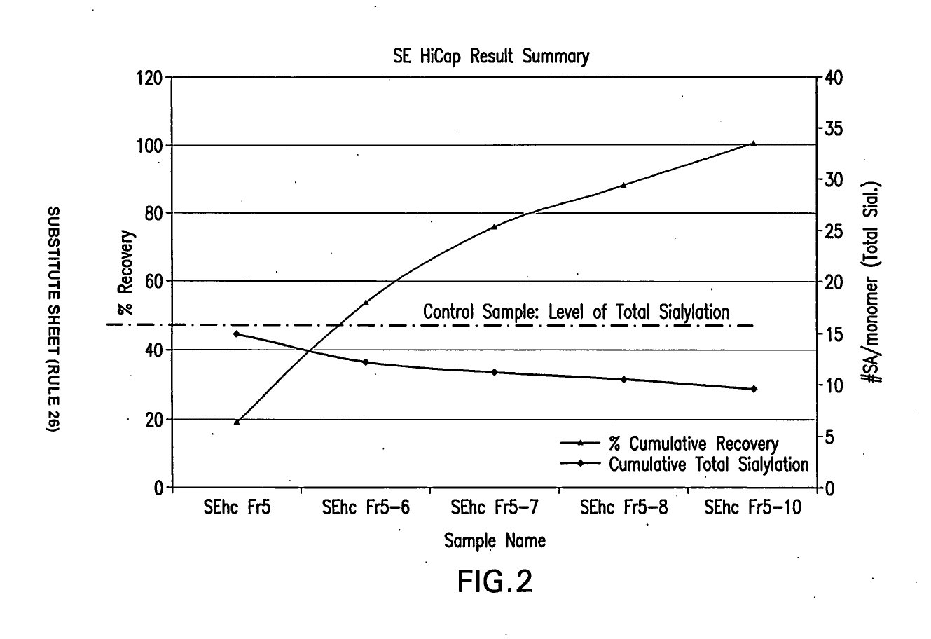

FIG. 1 shows the chromatogram obtained for the linear gradient run performed on SE HiCap. The results showed that SE HiCap provided limited enrichment of sialylation and TSA levels in all fractions were below the control sample level (FIG. 2).

4.1.2 Fractogel TMAE HiCap

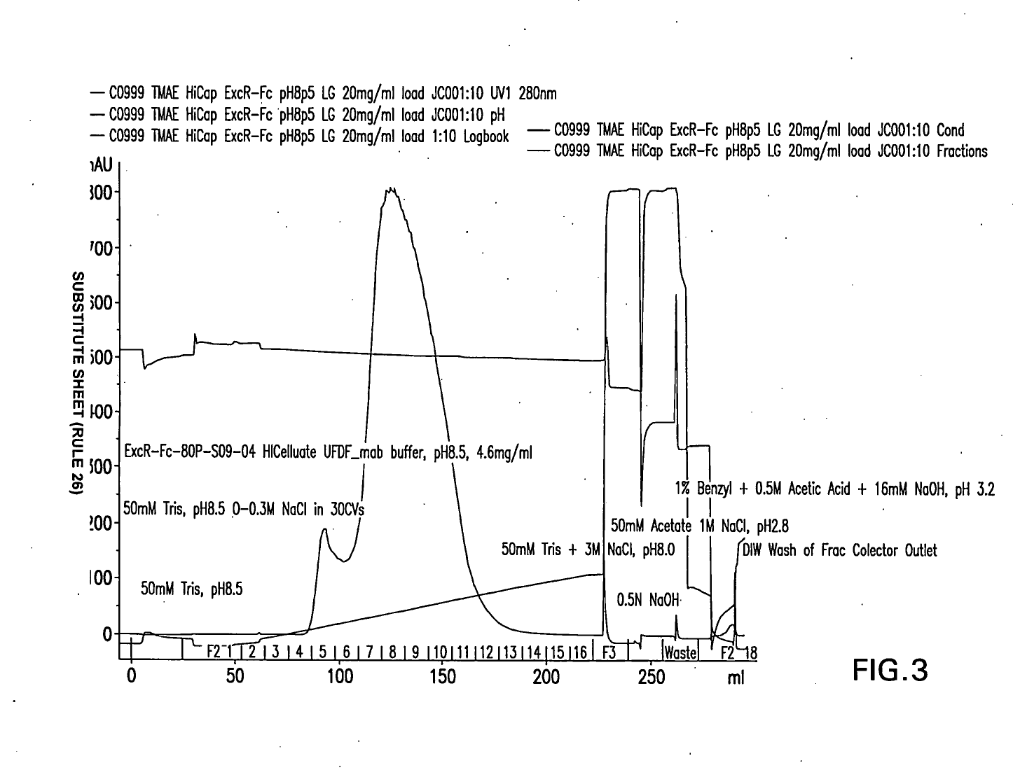

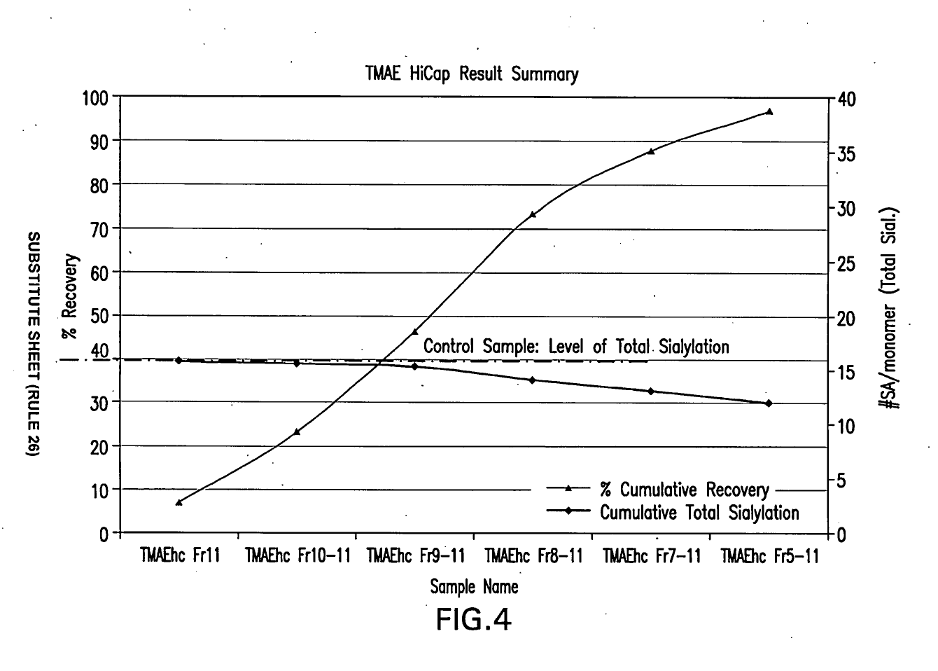

The linear gradient chromatogram on TMAE HiCap is shown in FIG. 3. TSA analysis of the eluate fractions showed that TMAE HiCap provided good resolution of sialylated product glycoforms (FIG. 4). As expected, the higher sialylated species bound stronger and eluting later in the gradient. Cumulative TSA levels for the later eluting fractions (i.e., Fraction 9 through 11) matched that of the control sample.

4.1.3 Capto DEAE

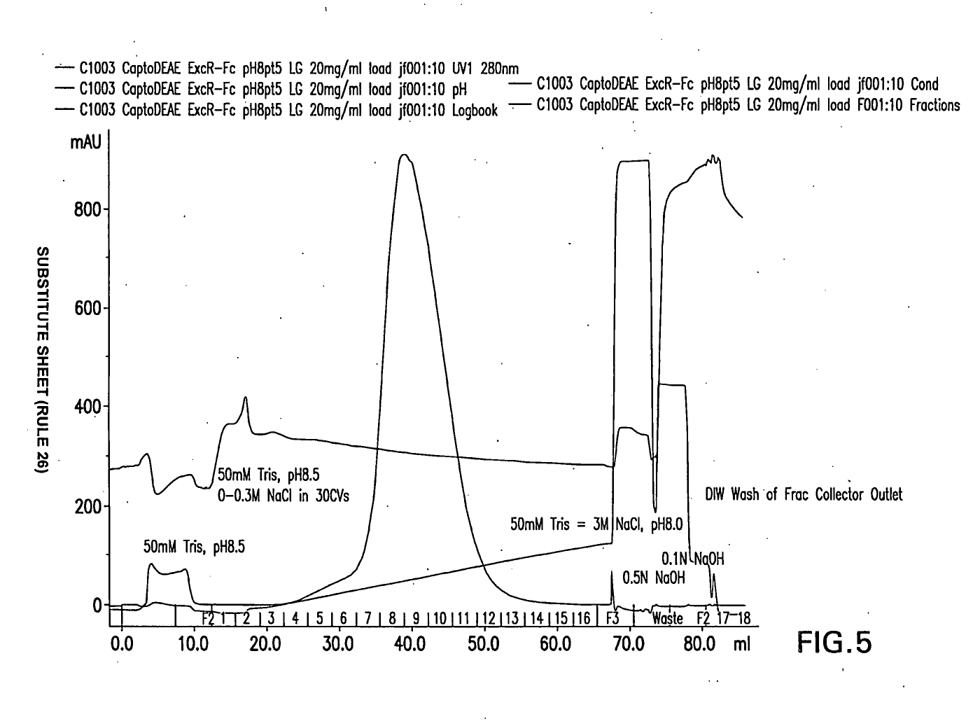

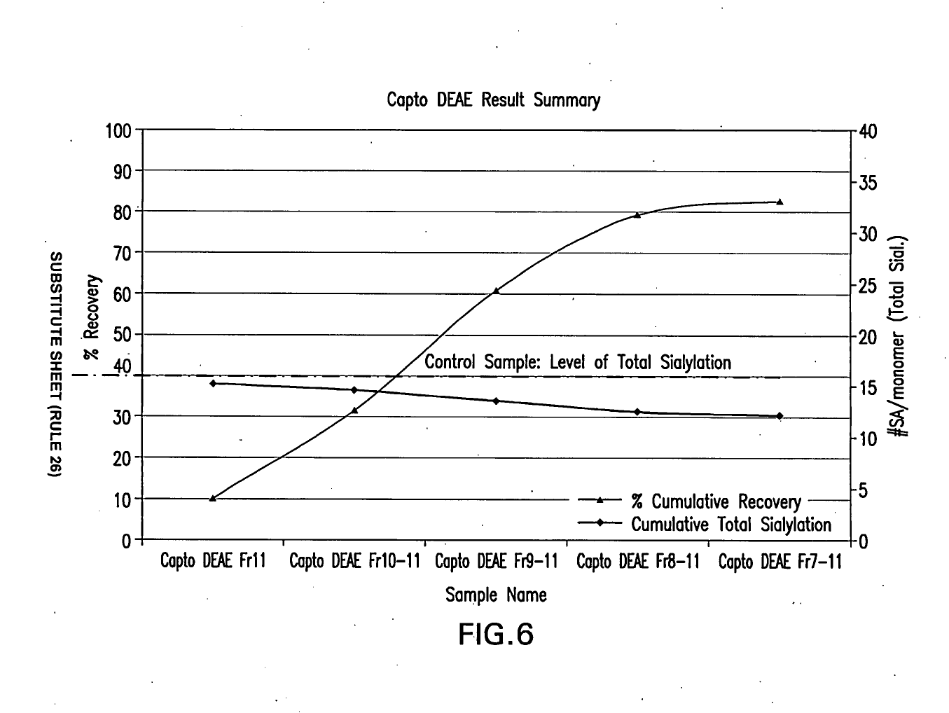

FIG. 5 shows the chromatogram for the linear gradient run performed on Capto DEAE. Analysis of the eluate fractions showed that this adsorbent provided limited separation of glycoforms (FIG. 6).

4.1.4 cHT Type I

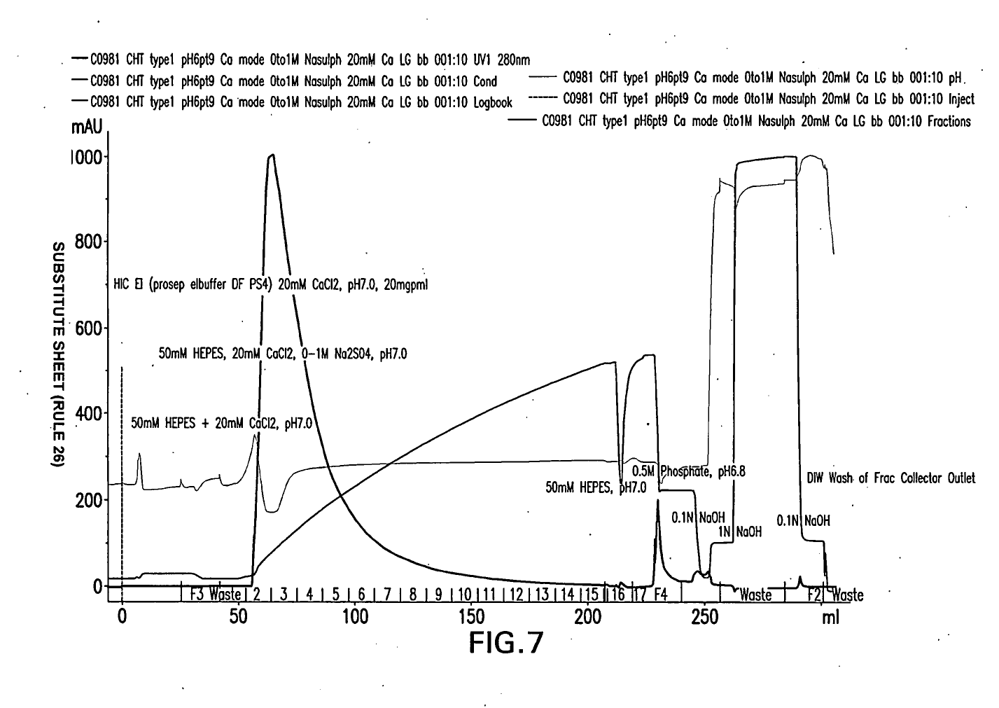

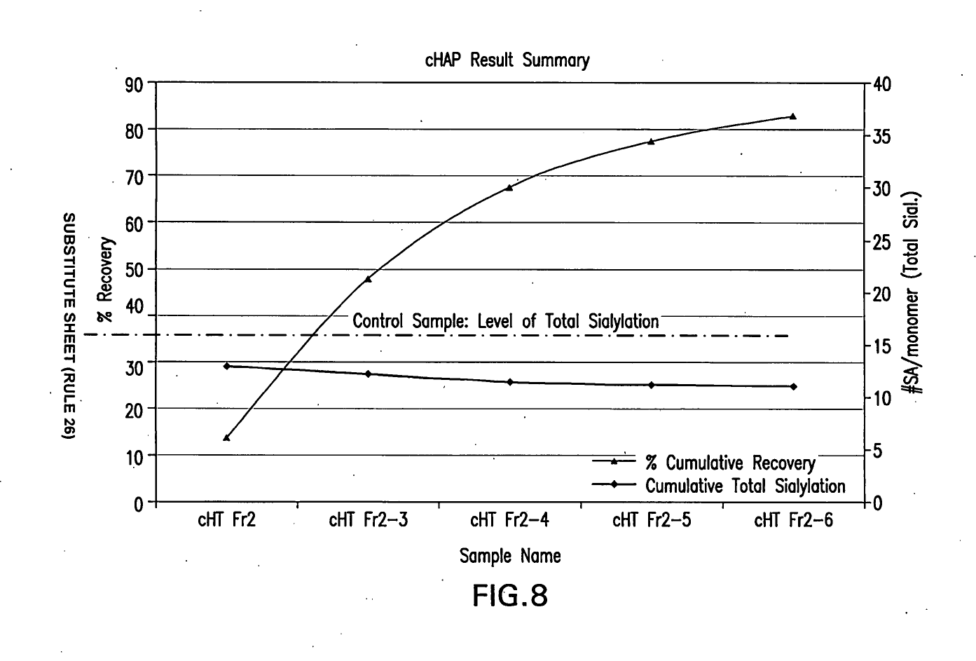

The linear gradient chromatogram for the experiment performed on cHT Type I is shown in FIG. 7. As shown in FIG. 8, this adsorbent provided almost no enhancement of product sialylation.

4.1.5 Phenyl Sepharose 6FF (high-sub)

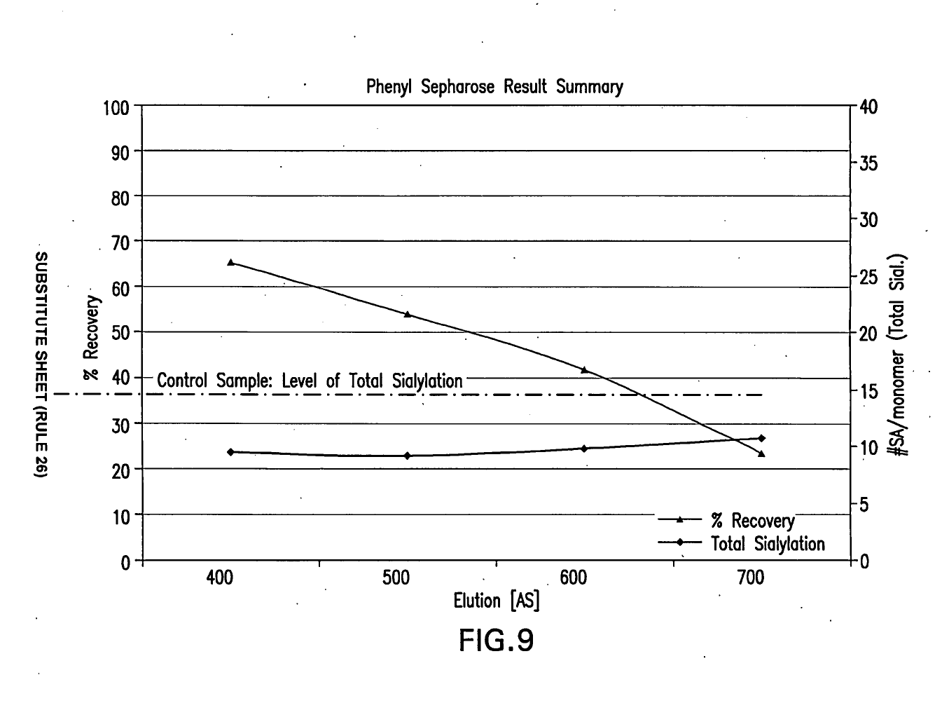

Eluate pools generated using different elution conditions between 400-700 mM ammonium sulfate during the course of development of the Phenyl Sepharose column as the third step in the ExcR-Fc purification process were analyzed by the TSA assay. As shown in FIG. 9, Phenyl Sepharose provided very limited resolution of various glycoforms.

4.1.6 Effect of pH on Glycoform Separation

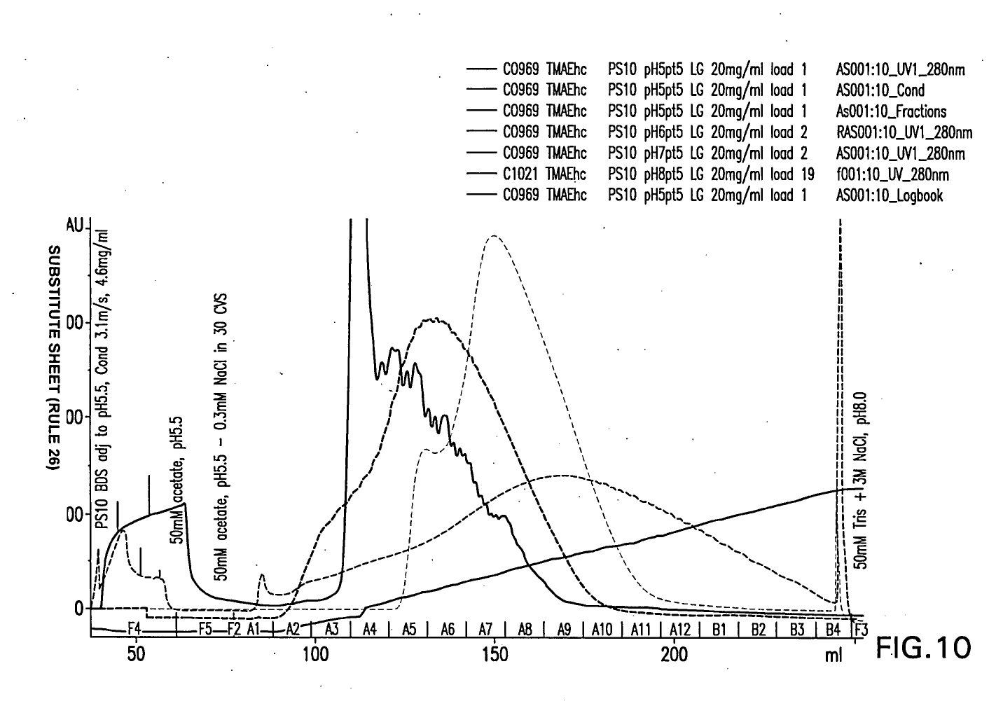

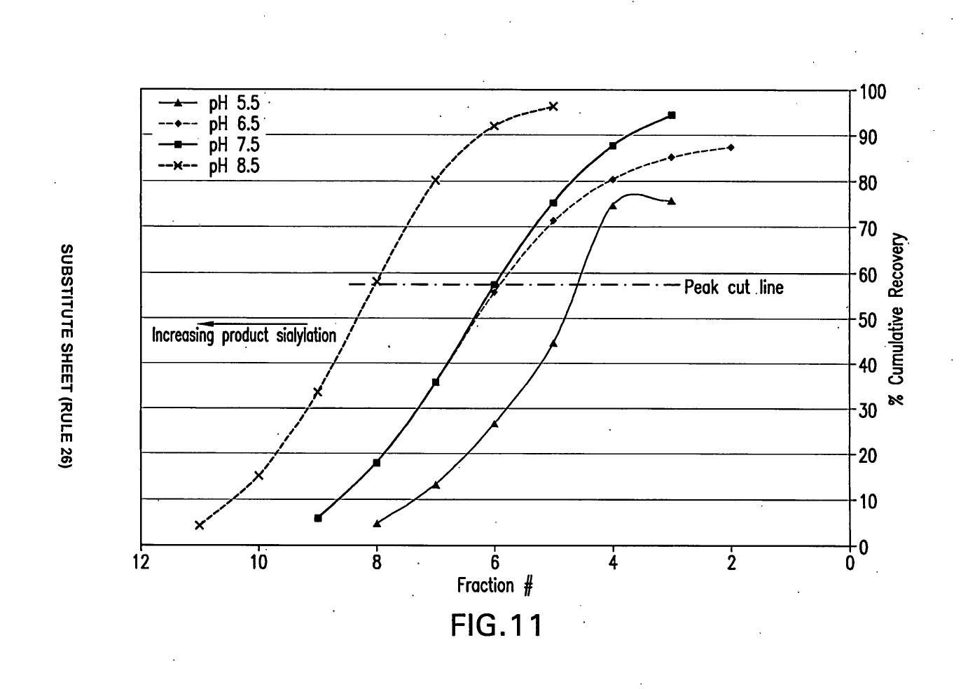

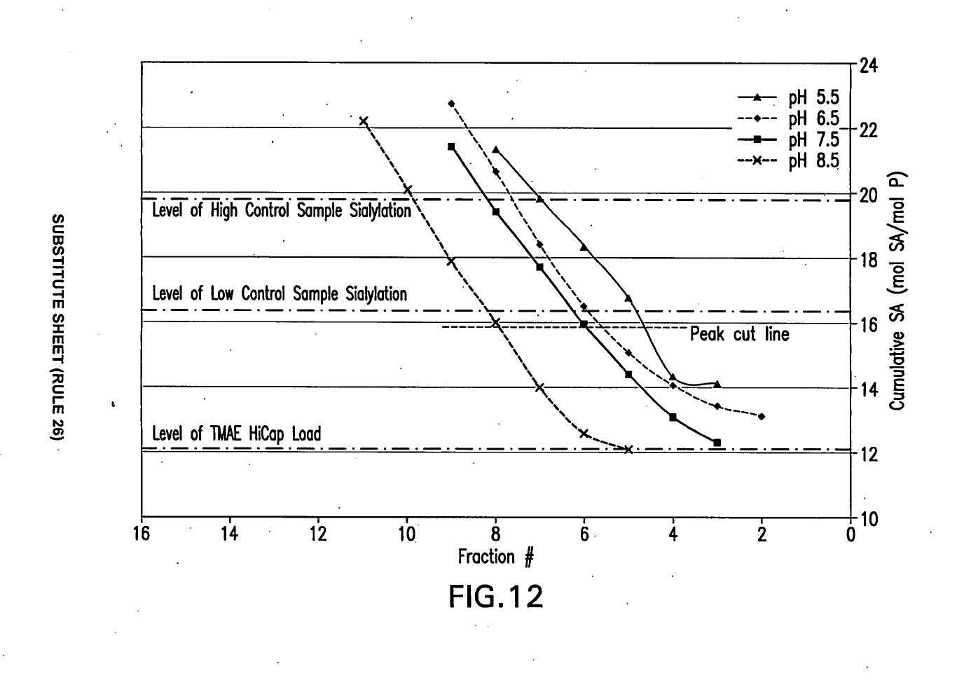

Additional linear gradient experiments were performed to evaluate the effect of pH on the separation of the glycoforms of ExcR-Fc on TMAE HiCap. To this end, experiments were performed at pH 5.5, 6.5, 7.5, and 8.5 and the eluate fractions analyzed by TSA analysis. FIG. 10 shows the overlaid chromatograms for these linear gradient runs. These results showed that comparable product yields (FIG. 11) and resolution of sialylated glycoforms (FIG. 12) were obtained at the different pH conditions as shown by the parallel curves in these plots. As expected, the level of product sialylation increased with increasing fraction number due to the greater binding affinity of higher sialylated glycoforms. Note: The curves at the different pH conditions were slightly offset from each other moving towards the higher fraction numbers with increasing pH due to the stronger binding on the TMAE HiCap adsorbent.

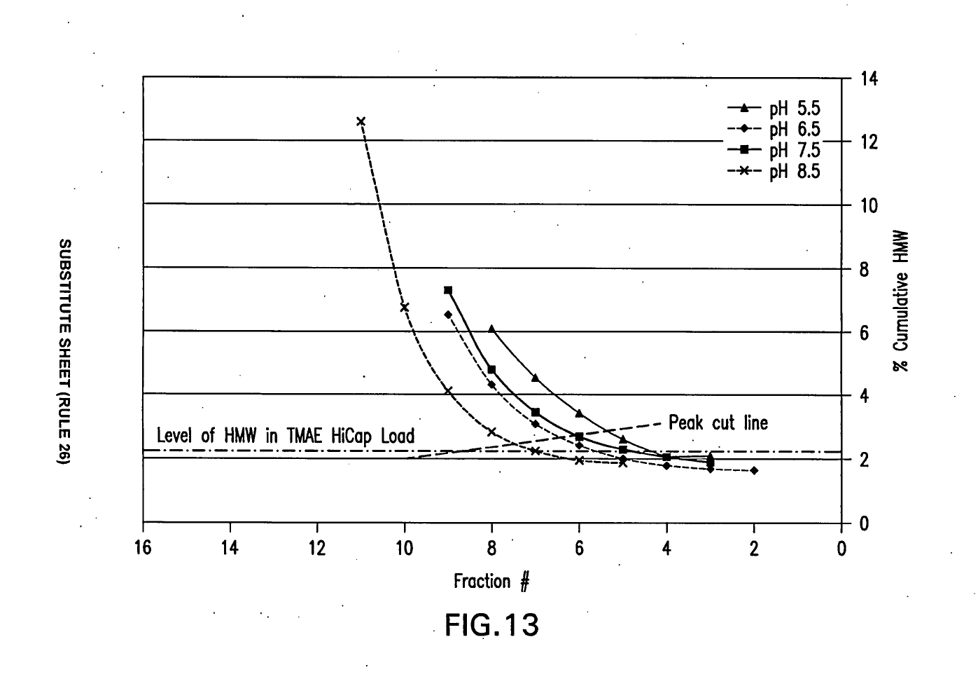

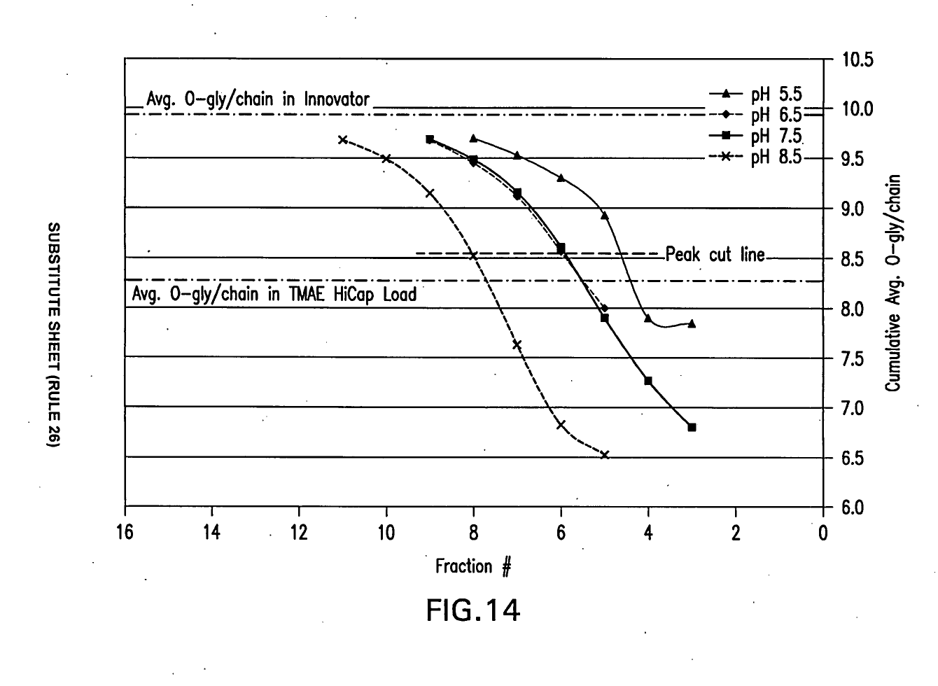

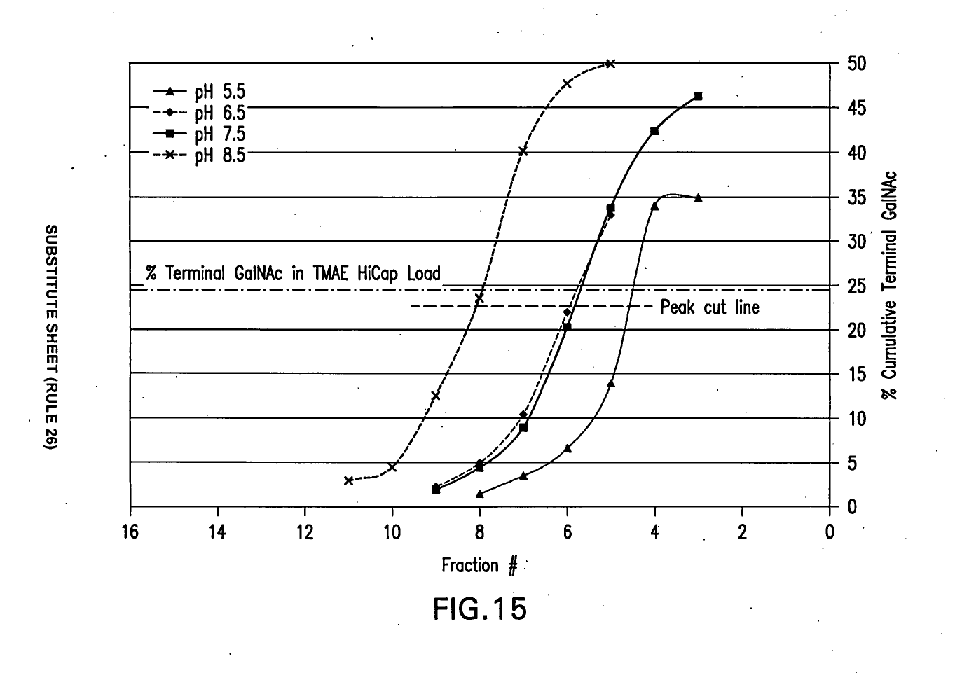

In addition, the eluate samples were also analyzed for HMW by SEC (FIG. 13), O-glycan occupancy by Intact Mass analyses (FIG. 14), and Terminal GalNAc by CE-LIF (FIG. 15). HMW levels increased with increasing fraction number—similar to trends seen for TSA—due to the higher binding affinity of HMW species on TMAE HiCap. Similarly, intact mass and CE-LIF results respectively showed that O-glycan occupancy increased and % terminal GalNAc decreased with increasing fraction number and approached the levels in the control sample material. Again, the relative changes in these product quality attributes were comparable at the different pH conditions as shown by the parallel curves in these figures. Thus, the TMAE HiCap step was able to enhance the TSA levels to match or exceed the level of the control sample material, while providing a concomitant improvement in the overall glycan profile of the product (O-glycan occupancy and Terminal GalNAc). At the same time, these results showed that no improvements in glycoform resolution and/or selectivity were obtained on TMAE HiCap by varying elution pH conditions.

### 4.2 Breakthrough and Partitioning Experiments

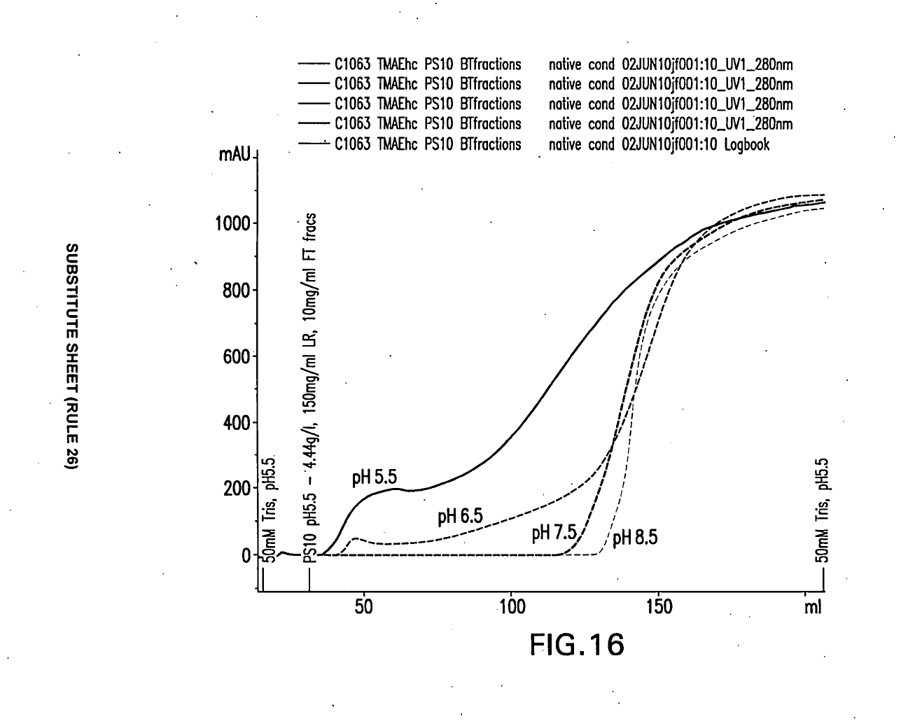

Breakthrough experiments were performed at different load pH conditions to evaluate the partitioning of differently sialylated product glycoforms on TMAE HiCap adsorbent. FIG. 16 shows the overlaid breakthrough curves obtained at different load pH. As shown in the figure, sharp product breakthrough was obtained at pH 7.5 and 8.5, while the shallower breakthrough curves were observed at pH 5.5 and 6.5 suggesting product partitioning between the stationary and mobile phases under these conditions. Table 3 shows the dynamic breakthrough capacity values (i.e., 10% DBC) calculated at the different load pH conditions.

TABLE 3
Dynamic Binding Capacity at Different Load Conditions

| | Load Conductivity | 10% DBC |
|---|---|---|
| Load pH | (mS/cm) | (mg/mL) |
| 5.5 | 3.16 | 6.0 |
| 6.5 | 3.42 | 54.8 |
| 7.5 | 3.46 | 76.9 |
| 8.5 | 3.43 | 85.5 |

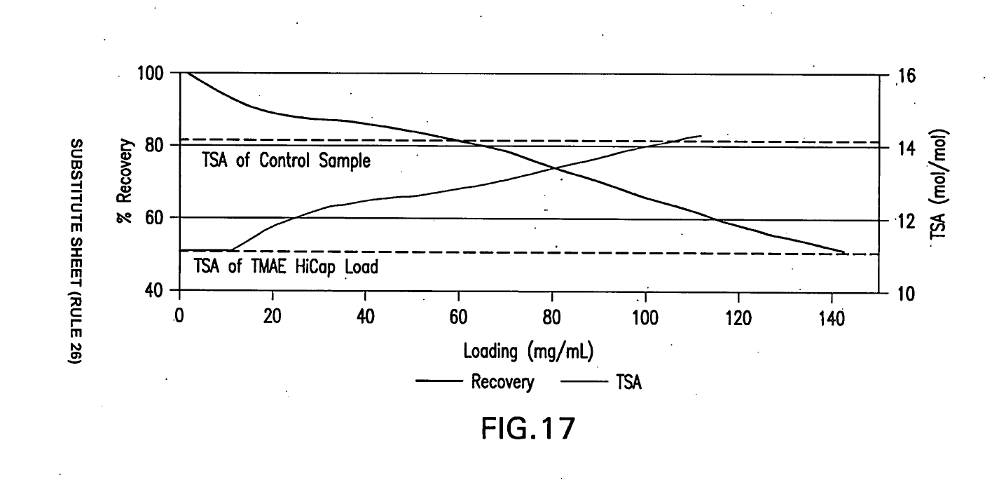

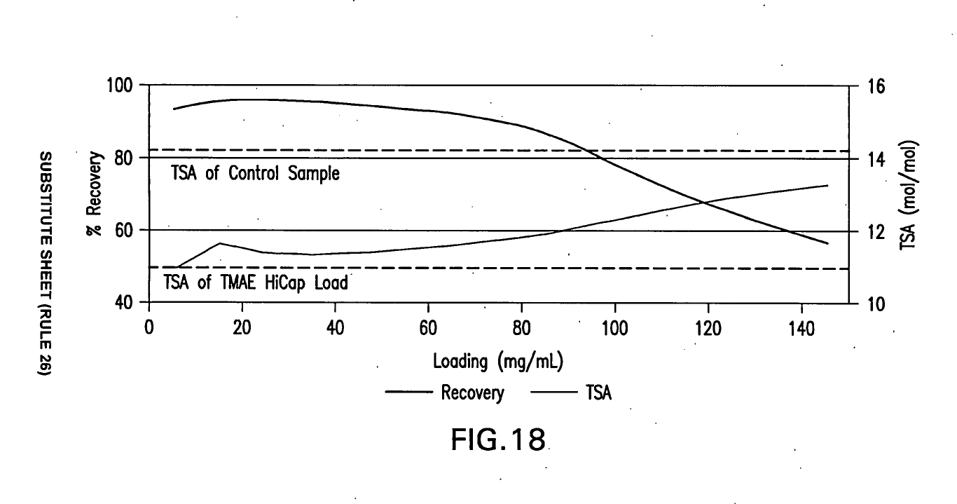

TSA results for the column flow through fractions for the runs at pH 5.5 and 6.5 showed that higher sialylated product forms, being more negatively charged, had a greater binding affinity to the TMAE HiCap adsorbent compared to the lower sialylated and non-sialylated species. Based on the product concentrations and TSA content of the column load and flow through fractions, a mass balance was performed to determine the mass (i.e., recovery) and TSA of the adsorbed product as a function of column loading (FIGS. 17 and 18). As shown in these figures, operating under these conditions offered a unique opportunity to enrich the TSA levels of the product by concentrating the sialylated glycoforms on the TMAE HiCap adsorbent by overloading the column and subsequently eluting the adsorbed protein from the column to obtain a highly sialylated product pool. The results also showed that significantly greater TSA enrichment at relatively higher product yield was obtained at pH 5.5 compared to the pH 6.5 load condition. Thus, the pH 5.5/˜3.2 mS/cm load condition at column loading≧100 mg/mL was selected for further development

### 4.3 Preliminary Development

Initial proof-of-concept experiments were performed to evaluate the ability of the TMAE HiCap column step to increase product sialylation by operating in the overloaded bind and elute modes under the loading conditions identified in the above breakthrough/partitioning studies i.e., pH 5.5/˜3.2 mS/cm. The results of the single point experiments performed at 100 and 150 mg/mL column loadings are summarized in Table 4. The results showed that when operated in this mode, the TMAE HiCap provided significant enrichment in sialylation, with the TSA levels being increased from 11.3 mol SA/mol ExcR-Fc in the load to 14.3-15.4 mol/mol in the eluate pools. In addition, eluate TSA levels increased with increasing loading with a concomitant drop in recovery due to higher product losses in the column flow through. Thus, the ability of the TMAE HiCap step to match the level of control sample sialylation (i.e., 14.6-15.2 mol/mol)) was successfully demonstrated. SEC analysis revealed that HMW levels were slightly increased from 2.2% in the load to 2.9-3.1% in the eluate pools. However, HMW levels in the column eluate were below the action limit of ≦3.5% for the release of biological product.

TABLE 4
Results of Proof-of-Concept Overloaded Bind and
Elute Experiments on TMAE HiCap

| Sample | Column | | Total | | | |
|---|---|---|---|---|---|---|
| De- | Loading | | Re- | Mass | | |
| scrip- | (mg/ | Load pH/ | covery | Balance | TSA | HMW |
| tion | mL) | Conductivity | (%) | (%) | (mol/mol) | (%) |
| Control | | | | | 14.6-15.2 | 1.4 |
| Sample | | | | | | |
| Load | | | | | 11.3 | 2.2 |
| Eluate | 100 | 5.5/ | 64.8 | 101.7 | 14.3 | 2.9 |
| | 150 | 3.14 mS/cm | 49.9 | 103.0 | 15.4 | 3.1 |

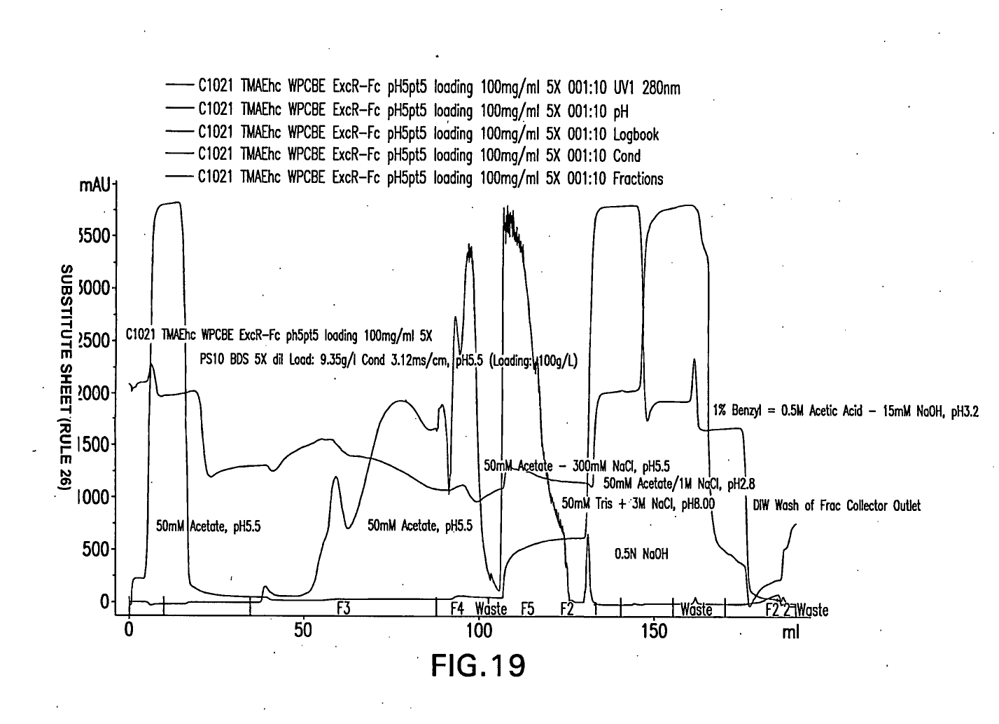

All previous TMAE HiCap development experiments employed a diafiltration step performed in a UF/DF system in order to prepare the TMAE HiCap load material from ExcR-Fc biological product generated in pilot-scale. In order to reduce the number of unit operations required for the reprocessing of the biological product to enhance sialylation, experiments were performed to evaluate the feasibility of employing diluted biological product as the column load material. As shown in FIG. 19, the experiment performed at a simple 5× dilution of the biological product showed an atypical and irregular load breakthrough curve suggesting potential interference from formulation buffer components and/or lack of pH buffering capacity in the load material. An atypical breakthrough curve as well as significant product binding (due to lower load conductivity) were obtained when the biological product was diluted 10× with WFI (data not shown). Owing to the lack of process control under these conditions, these options were dropped from consideration and the samples were not submitted for analytical testing.

The experiment performed with biological product diluted 5× with WFI followed by a 2× dilution with buffer (50 mM acetate, pH 5.5) showed more typical breakthrough behavior (chromatogram not shown, 6). In addition, the TSA enrichment (relative to the load TSA) obtained in this case was comparable to that obtained with diafiltered biological product at comparable column loading and load pH and conductivity (i.e., 3.1 mol/mol enrichment, Table 5 vs. 3.0 mol/mol enrichment, Table 4). However, greater product binding and consequently lower yield was obtained in this case relative to the run performed using diafiltered biological product (i.e., 56%, Table 5 vs. 65%, Table 4). Thus, the above results clearly demonstrate the need for the UF/DF step for diafiltering the ExcR-Fc biological product into the appropriate load buffer to ensure better process control and higher product recovery from the TMAE HiCap step.

TABLE 5
Effect of Load Composition

| Sample | | Column | Load pH/ | Re- | TSA | |
|---|---|---|---|---|---|---|
| De- | Load | Loading | Cond. | covery | (mol/ | HMW |
| scription | Dilution | (mg/mL) | (mS/cm) | (%) | mol) | (%) |
| Control | | | | | 15.8 | 1.4 |
| Sample | | | | | | |
| Load | | | | | 12.2 | 2.7 |
| Eluate | 5X | 100 | 5.5/3.12 | n.d. | n.d. | 3.0 |
| | 10X | | 5.5/1.64 | n.d. | n.d. | 3.3 |
| | 5X WFI + | | 5.5/3.31 | 55.5 | 15.3 | 3.7 |
| | 2X Buffer | | | | | |

n.d.: Not determined

4.4 Design of Experiments (DoE) Study

Table 6 shows the results of the DoE experiments performed to determine suitable conditions for the operation of the TMAE HiCap overloaded bind and elute chromatography process. TSA, HMW, and recovery were measured and modeled as the key process outputs. Host cell protein (HCP) levels and product purity by reduced and non-reduced LC90/GXII were also monitored to ensure that all other purity targets were adequately controlled by the process. As shown in the table, the eluate pools of all DoE runs had comparable and high purity as well as very low levels of HCP i.e., at or near the assay LOQ.

TABLE 6
Results of DoE Experiments

| | | | | | Non-reduced LC90 | | |
|---|---|---|---|---|---|---|---|
| | | | | | % | | Reduced |
| | | | | | Highest | % Purity | LC90 |
| | Recovery | HMW | TSA | HCP | Single | of Main | % Purity of |
| Run | (%) | (%) | (mol/mol) | (ppm) | Impurity | Peak | Main Peak |
| LOAD | — | 0.90 | 14.0 | <1.7 | 1.5 | 98.2 | 99.1 |
| 1 | 65.4 | 1.24 | 16.5 | 0.6 | 0.9 | 98.6 | 99.1 |
| 2 | 56.0 | 1.26 | 17.2 | <0.6 | 0.7 | 98.9 | 99.1 |
| 3 | 70.0 | 1.04 | 15.9 | 0.6 | 1.1 | 98.6 | 99.2 |
| 4 | 55.0 | 1.26 | 16.9 | 1.4 | 3.3 | 95.6 | 98.3 |
| 5 | 65.8 | 1.09 | 16.5 | 0.6 | 0.8 | 98.8 | 99.2 |
| 6 | 84.5 | 1.15 | 15.7 | 0.8 | 2.2 | 97.3 | 98.7 |
| 7 | 38.8 | 1.46 | 17.7 | 0.6 | 0.9 | 98.5 | 99.0 |
| 8 | 64.3 | 1.15 | 16.6 | 0.6 | 0.9 | 98.7 | 99.1 |
| 9 | 49.2 | 1.31 | 17.5 | 0.8 | 0.7 | 99.0 | 99.1 |
| 10 | 50.4 | 1.23 | 17.0 | 0.7 | 0.8 | 98.9 | 99.1 |
| 11 | 77.8 | 1.09 | 16.0 | 0.6 | 1.0 | 98.7 | 99.1 |
| 12 | 59.5 | 1.25 | 17.0 | <0.5 | 0.7 | 98.9 | 99.1 |
| 13 | 63.6 | 0.92 | 15.7 | 0.6 | 1.3 | 98.1 | 99.2 |
| 14 | 99.3 | 0.98 | 14.4 | <0.5 | 1.5 | 98.2 | 99.2 |
| 15 | 76.2 | 1.03 | 15.6 | 0.6 | 1.1 | 98.6 | 99.2 |
| 16 | 65.2 | 1.09 | 16.5 | <0.6 | 0.9 | 98.7 | 99.2 |
| 17 | 74.4 | 0.99 | 15.9 | 0.5 | 1.2 | 98.4 | 99.0 |
| 18 | 54.7 | 1.29 | 17.2 | 0.6 | 0.7 | 98.9 | 99.0 |

Note:
TSA for the control sample employed during the analysis of the DoE samples was between 16.0-16.5 mol/mol.

Results from experimental runs were imported into Stat-Ease Design Expert v8.0 software and analyzed. Analysis of each output involved the selection of a mathematical model that described the effect of process parameters on that output. The data were fit by the best model equation and an analysis of variance (ANOVA) was performed to remove any insignificant parameters. This is outlined for each output in subsequent sections. The following methodology was utilized for analysis of each response:

The fit summary program was exploited to perform regression calculations to fit all of the polynomial models to the selected response. The fit summary program calculated the effects for all model terms and produced P-values, lack of fit, and R² statistics in order to compare and select the best model(s). The model selected was the highest order polynomial where the additional terms were significant, the model was not aliased, lack of fit was minimized and adjusted and predicted R² values were maximized.

Significant parameters were determined using analysis of variance for each response by removing input parameters one at a time via backward selection until only process variables remained if there was less than a 5% chance that their effect on a response could be due to noise alone (probability of a larger F-value [Prob>F]<0.05; p-value<0.05). These factors were deemed to be significant factors affecting the response. Factors were included in the model when P-values≧0.05 to support hierarchy.

Additional analysis of variance was examined to ensure that the model was significant and lack of fit was insignificant (p-values≧0.05) and to examine adjusted and predicted R² values. A predicted R² value of ≧0.5 was utilized to determine if a model was adequate for process modeling. The predicted and adjusted R² values should be within 0.2 of each other.

Diagnostic tools were utilized to examine if the data contained outliers, non-normality or heteroskedasticity of residuals or required a transformation. After transformations were applied and outliers investigated, significant factors were identified and models were generated.

Table 7 summarizes results from the analysis of variance which identifies significant factors that affect each response (p-value<0.05) and examines model fit and prediction capabilities through sum of squared error calculations and lack of fit significance. No significant model or significant parameters were identified that affected HCP and % purity. The analysis of the individual responses can be found in Sections 5.4.1-5.4.6.

TABLE 7
ANOVA Summary for the TMAE HiCap DoE Study

| Input | Ranges | TSA | | Recovery | |
|---|---|---|---|---|---|
| Parameter | Tested | (mol/mol) | HMW (%) | (%) | |
| A: Loading | 90-160 | <0.0001 | 0.0050 | <0.0001 | p-values |
| (mg/mL) | | | | | |
| B: Load pH | 5.2-5.8 | <0.0001 | 0.0001 | <0.0001 | |
| C: Load | 2.4-4.4 | <0.0001 | <0.0001 | <0.0001 | |
| Conductivity | | | | | |
| (mS/cm) | | | | | |
| AB: Loading * | — | 0.0468 | * | 0.0086 | |
| Load pH | | | | | |
| AC: Loading * | — | * | * | 0.0040 | |
| Load | | | | | |
| Conductivity | | | | | |
| BC: Load pH * | — | 0.0073 | * | * | |
| Load | | | | | |
| Conductivity | | | | | |
| A\hat{ }2: Loading * | — | 0.0066 | * | * | |
| Loading | | | | | |
| Lack of Fit | | 0.1478 | 0.6488 | 0.0516 | |
| R² | | 0.9839 | 0.8264 | 0.9911 | |
| Adjusted R² | | 0.9751 | 0.7892 | 0.9874 | |
| Predicted R² | | 0.9161 | 0.6629 | 0.9702 | |
| Adequate | | 44.28 | 18.60 | 65.99 | |
| Precision | | | | | |
| Transformation | | None | None | None | |

* p-values > 0.05 are not shown unless required to support hierarchy.

A model was produced for each response in the form of the following equation. Coefficients for each response are listed in Table 8.

Response=B₀+A(Loading)+B(Load pH)+C(Load Conductivity)+D(Loading*Load pH)+E(Loading*Load Conductivity)+F(Load pH*Load Conductivity)+G(Loading)²

TABLE 8
Coefficients for the Predictive Response Surface Models

| Response | B₀ | A | B | C | D | E | F | G |
|---|---|---|---|---|---|---|---|---|
| TSA | 34.60 | 5.94E−03 | −4.57 | −2.08 | 9.73E−03 | — | 0.499 | −1.67E−04 |
| (mol/mol) | | | | | | | | |
| HMW | 2.49 | 1.95E−03 | −0.354 | 0.110 | — | — | — | — |
| (%) | | | | | | | | |
| Recovery | −64.26 | 0.330 | 43.00 | −16.81 | −0.168 | 0.057 | — | — |
| (%) | | | | | | | | |

For the TSA, HMW, and Recovery responses, 99% prediction intervals were generated for a given combination of input parameters within the operating space to predict the corresponding reponse levels. For the reponses for which no model was identified i.e., HCP, Highest Single Impurity and Purity, three standard deviations around the mean value were utilized to predict response levels (Table 9).

TABLE 9
Predicted Response Levels for Responses with No Models

| | | | | Predicted |
|---|---|---|---|---|
| | | | | Response |
| Response | Mean | σ | 3σ | Values |
| HCP (ppm) | | | | <1.7 (LOQ) |
| Highest Single Impurity (%) | 1.2 | 0.6 | 1.9 | 0-3.1 |
| Purity (%) (Non-Red. LC90) | 98.4 | 0.8 | 2.4 | 96.0-100.0 |
| Purity (%) (Red. LC90) | 99.1 | 0.2 | 0.7 | 98.4-99.8 |

4.4.1 Total Sialic Acid (TSA)

The enhancement of TSA is the key function of the TMAE HiCap chromatography step in the ExcR-Fc downstream purification process. Thus, the TSA level in the eluate pool was the key response for the design and modeling of this step. The Fit Summary of these data is shown in Tables 10-12. The sequential model sum of squares table (Table 10) shows the cumulative improvement in the model fit as higher order model terms are added. For example, Linear vs. Block shows the significance of the linear terms after accounting for the linear and block terms. Similarly, 2FI vs. Linear gives the significance of adding two-factor interaction terms to the model; Quadratic vs. 2FI indicates the significance of adding the quadratic terms to the linear, block, and the 2FI terms; and so forth. Each row in the table contains the statistics for additional terms only and not for the complete model.

The significance associated with the addition of model terms is calculated using the sum of squares (SS) for the model as well as the residual error. The sum of squares for all effects which were not included in the model were pooled together and used as an estimate of the residual error. The mean square error was calculated by dividing the sum of squares by the degrees of freedom (SS/df). The ratio of the mean squares (MSModel/MSResidual) was used to determine the F-value for the model, which was then used to compare the variance of the model with the variance of the residual error. The larger the F-value, the greater the likelihood of the model being significant. A quantitative measure of model significance can be obtained by comparing the F-value to known F-distribution tables for a given percentage risk. F-values greater than the critical value given on these tables are considered significant within the stated percentage risk. In addition, the Probability>F value (i.e., p-value) was calculated. This value is equal to the fractional area under the curve of the F-distribution that lies beyond the observed F-value. For example, a p-value of <0.05 indicates that there is 95% confidence that the model is significant and not due to experimental noise. In general, model terms having a p-value of <0.05 are deemed to be significant. The highest order model where the additional terms were significant and where aliasing was not present was indicated in the table as the “suggested” model. Analysis of the TSA data suggested that a quadratic model best described this response (Table 10).

TABLE 10
Sequential Model Sum of Squares for TSA

| | Sum of | | Mean | | p-value | |
|---|---|---|---|---|---|---|
| Source | Squares | df | Square | F Value | Prob > F | Comments |
| Mean vs | 4861.593 | 1 | 4861.593 | | | |
| Total | | | | | | |
| Linear vs | 10.727 | 3 | 3.576 | 79.32 | <0.0001 | |
| Mean | | | | | | |
| 2FI vs | 0.2788 | 3 | 0.093 | 2.89 | 0.0837 | |
| Linear | | | | | | |
| Quadratic | 0.196 | 3 | 0.065 | 3.33 | 0.0770 | Suggested |
| vs 2FI | | | | | | |
| Cubic vs | 0.136 | 4 | 0.034 | 6.65 | 0.0468 | Aliased |
| Quadratic | | | | | | |
| Residual | 0.021 | 4 | 0.005 | | | |
| Total | 4872.95 | 18 | 270.720 | | | |

The second table compiled in the fit summary is the lack of fit tests table (Table 11). This table compares how well each model fits the data. The lack of fit test compares the residual error to the pure error from replicated design points. A lack of fit error significantly larger than the pure error indicates that there are experimental points that differ significantly from the values predicted by the model. Accordingly, a more appropriate model should be used fit to the data. The p-value is used to measure the lack of fit. The higher the p-value, the greater the likelihood that the difference between the experimental and model points is due to experimental noise. In general, a p-value greater than 0.1 is desired. For the TSA response, the quadratic model had the highest p-value for the non-aliased models, suggesting it had the best fit to the data.

TABLE 11
Lack of Fit Tests for TSA

| | Sum of | | Mean | | p-value | |
|---|---|---|---|---|---|---|
| Source | Squares | Df | Square | F Value | Prob > F | Comments |
| Linear | 0.615 | 11 | 0.056 | 10.30 | 0.0400 | |
| 2FI | 0.337 | 8 | 0.042 | 7.76 | 0.0598 | |
| Quadratic | 0.141 | 5 | 0.028 | 5.18 | 0.1031 | Suggested |
| Cubic | 0.004 | 1 | 0.004 | 0.78 | 0.4421 | Aliased |
| Pure Error | 0.016 | 3 | 0.005 | | | |

Table 12 contains the model summary statistics for the TSA response. The R-squared, adjusted R-squared, predicted R-squared, and the PRESS statistic for each complete model type are shown. As shown in the table, the quadratic model had the highest R-squared and adjusted R-squared values, which indicated that it best fit the experimental data.

TABLE 12
Model Summary for TSA

| | Std. | R- | Adjusted | Predicted | | |
|---|---|---|---|---|---|---|
| Source | Dev. | Squared | R-Squared | R-Squared | PRESS | Comments |
| Linear | 0.212 | 1 | 0.933 | 0.89 | 1.269 | |
| 2FI | 0.179 | 1 | 0.952 | 0.82 | 2.004 | |
| Quadratic | 0.140 | 1 | 0.971 | 0.81 | 2.209 | Suggested |
| Cubic | 0.072 | 1 | 0.992 | 0.47 | 5.983 | Aliased |

The quadratic model for TSA was then reduced to remove any model terms that were not significant. This was done by using a backward selection process, involving the calculation of the ANOVA (analysis of variance) for the model, removing the least significant model term, calculating the ANOVA for the model with the term removed, and again removing the least significant term. This process was repeated until all model terms having a p-value>0.05 were removed from the model. The ANOVA results for the eluate TSA model is shown in Table 13.

TABLE 13
ANOVA Table for TSA

| | p-value | |
|---|---|---|
| Source | Prob > F | Comments |
| Model | <0.0001 | Significant |
| A-Loading | <0.0001 | |
| B-Load pH | <0.0001 | |
| C-Load Conductivity | <0.0001 | |
| AB | 0.0468 | |
| BC | 0.0073 | |
| A\hat{ }2 | 0.0066 | |
| Lack of Fit | 0.1478 | not significant |
| R-Squared | 0.9839 | |
| Adj R-Squared | 0.9751 | |
| Pred R-Squared | 0.9161 | |
| Adeq Precision | 44.28 | |

The ANOVA results showed that the model for TSA had high R² values (>0.9) indicating that the model captured more than 90% of the variance in the data. In addition, the Prediction R² value was in close agreement with the Adjusted R² value, suggesting that this model had good predictive ability. The Adequate Precision, which measures the signal to noise ratio, had a value of 44.28 indicating that the model had adequate signal and may be used to navigate the design space.

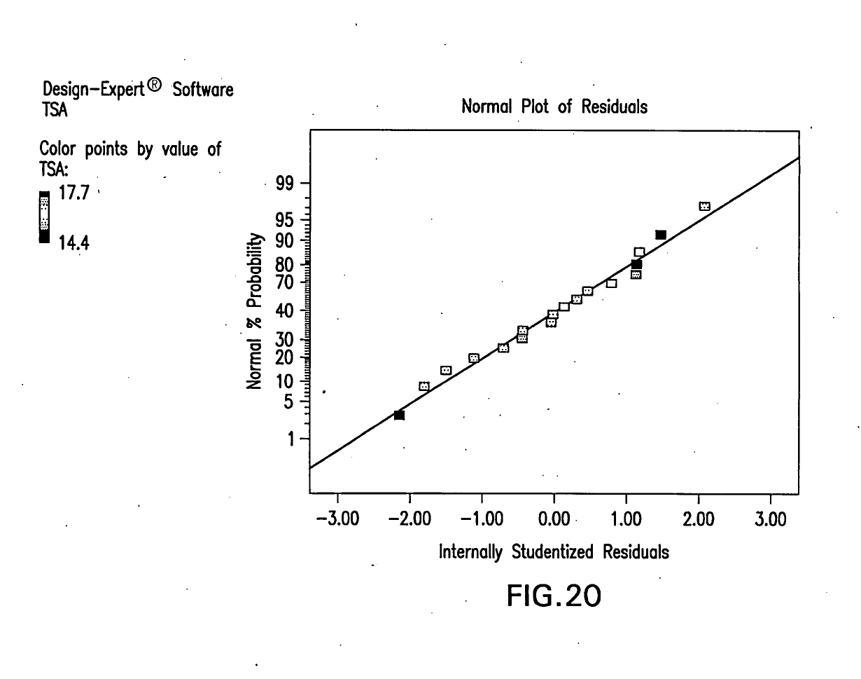

In order to validate the assumptions of the ANOVA (normal distribution of residuals, independence of errors, and constant variance) a number of diagnostic plots were evaluated. All residuals in the diagnostic plots were studentized (i.e., residuals were divided by their estimated standard deviation) for the purpose of comparison. A normal distribution plot (FIG. 20) indicates whether the residuals are normally distributed. As shown in the figure, the residuals for the TSA response appeared normally distributed and there was no distinctive pattern found in the data.

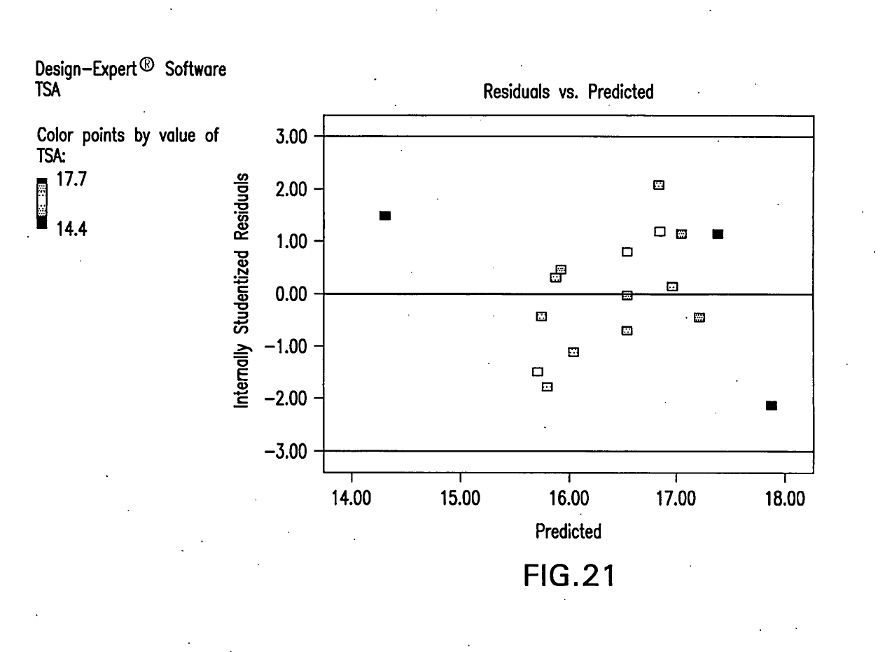

FIG. 21 shows the plot of the studentized residuals vs. the predicted values examines the assumption of constant variance in the ANOVA calculations. This plot for the TSA response showed no significant trends, which confirmed the validity of the assumption of constant variance in the ANOVA calculations.

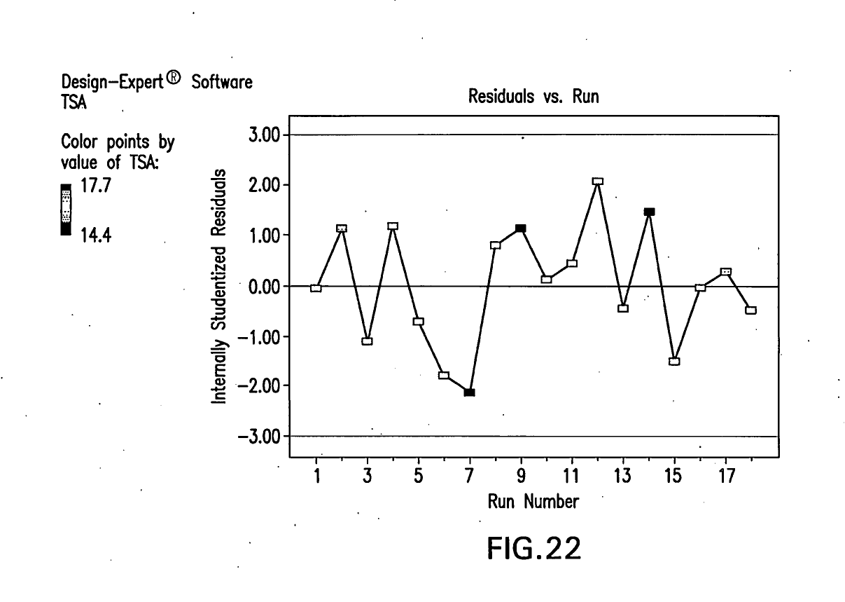

The lack of trending in the TSA residuals vs. run number plot (FIG. 22) indicated that there were no lurking variables that significantly impacted the results.

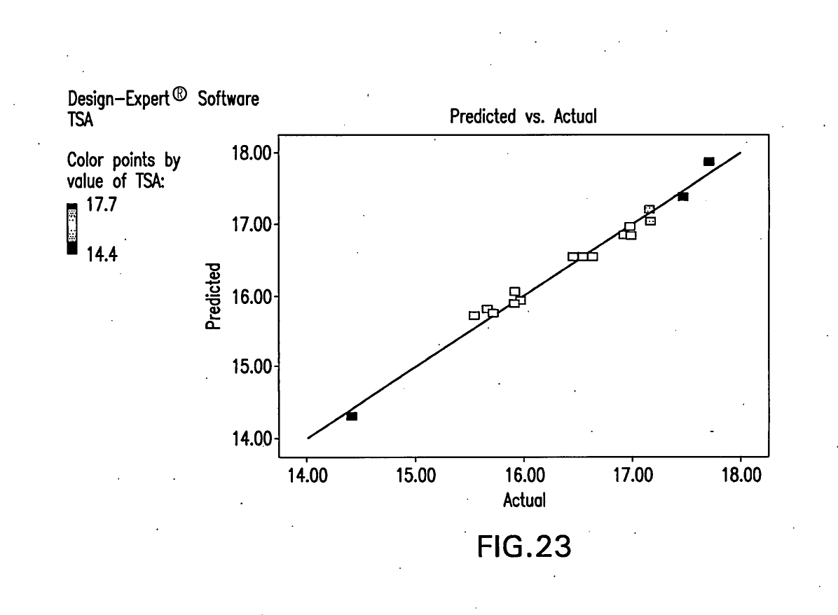

The graph of the predicted eluate TSA levels vs. the actual values enables the (FIG. 23) shows that within the explored ranges for the screening DOE, there was reasonable agreement between the experimental and predicted values for the TSA levels in the TMAE HiCap eluate.

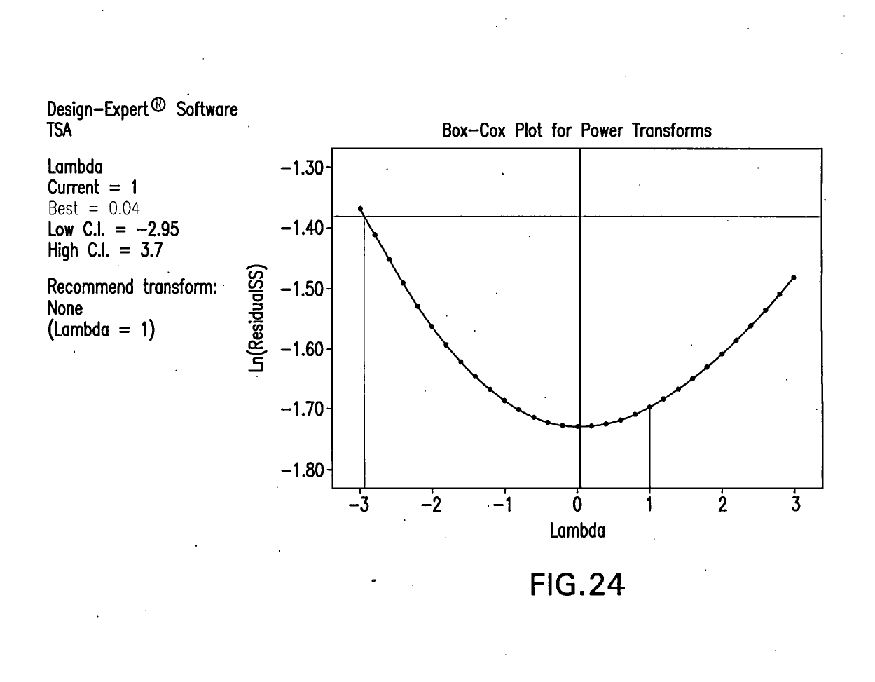

FIG. 24 shows the Box Cox response of the TSA response. The vertical blue line in the Box-Cox plot corresponds to power transformation of current data (lambda is a parameter of the transformation). The vertical green line corresponds to the transformation that would result in the lowest SSE. A 95% confidence interval was also calculated, which is shown on the plot by the vertical red lines on either side of best-fit line. If the current transformation is within the 95% confidence of the best-fit, then a transformation is not recommended. As shown in the figure, the TSA data were found to be normally distributed. Therefore, no transformation was recommended.

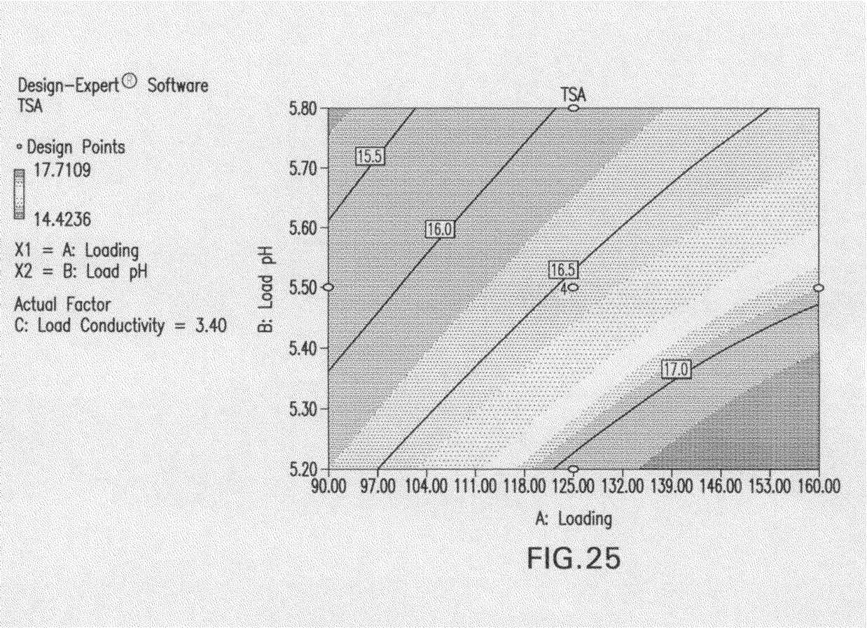

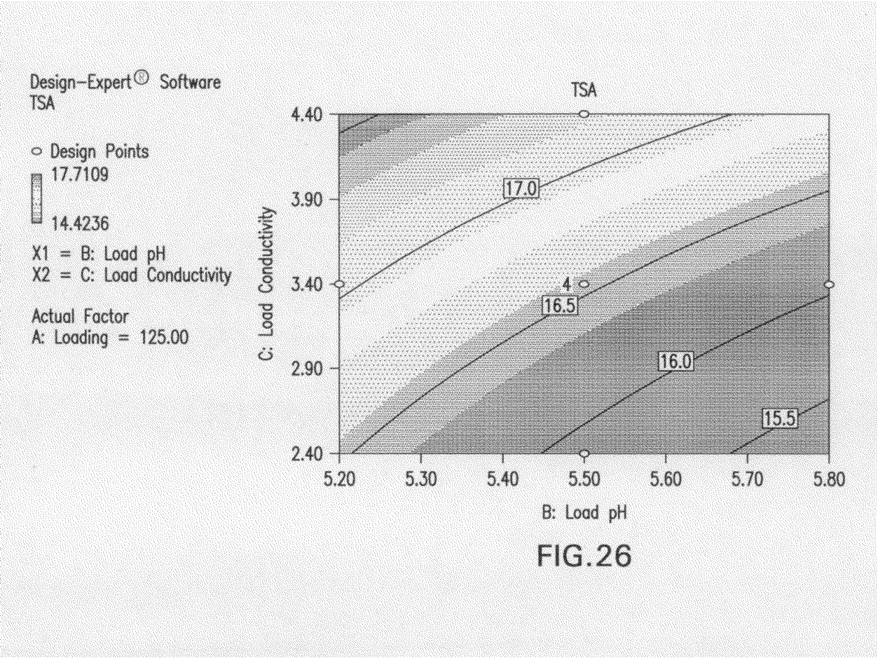

FIGS. 25 and 26 show the contour plots generated by the Design Expert software showing the change in eluate TSA level as a function of the key process parameters—column loading, load pH and load conductivity—for the TMAE HiCap overloaded bind and elute chromatography step. As shown in these figures, TSA levels increased with increasing loading and/or with decreasing load pH and/or with increasing load conductivity.

4.4.2 HMW

Previous development results showed that the TMAE HiCap eluate had higher HMW levels than the corresponding load material. This was expected due to the higher binding affinity of HMW relative to monomer, resulting in the accumulation and concentration of HMW on the adsorbent and correspondingly higher levels in the eluate. This behavior is analogous to that shown by the higher sialylated glycoforms and leads to the co-enrichment of HMW species. Thus, eluate HMW is another key process output that must be modeled and characterized and controlled (if required).

Analysis of HMW results for the TMAE HiCap eluate pools within the Design Expert software suggested that a linear model best described this response (tables not shown, 7). After applying the backward selection with alpha=0.05 to identify the significant factors, the ANOVA table for the model identified A, B, and C as significant factors (Table 14).

TABLE 14
ANOVA Table for HMW

| | p-value | |
|---|---|---|
| Source | Prob > F | Comments |
| Model | <0.0001 | Significant |
| A-Loading | 0.0050 | |
| B-Load pH | 0.0001 | |
| C-Load Conductivity | <0.0001 | |
| Lack of Fit | 0.1478 | not significant |
| R-Squared | 0.9839 | |
| Adj R-Squared | 0.9751 | |
| Pred R-Squared | 0.9161 | |
| Adeq Precision | 44.28 | |

The lack of fit for the above model was insignificant and the model had high R² and adjusted R² values. Also, the predicted R² value was in good agreement with the adjusted R² indicating that the model had good predictive ability. Diagnostic plots revealed that the distribution of residuals was normal, the variance was constant, and there was no influence by lurking variables on the response, which confirmed that the ANOVA was valid (plots not shown, 7). Analysis of the model plots (not shown, 7) showed that HMW levels in the TMAE eluate increased with increasing column loading, decreasing load pH, and/or increasing load conductivity. Since the load material employed for these experiments had ˜0.9% HMW, the eluate HMW levels for all runs were well below the action limit of >3.5% HMW (Table 6). Thus there were additional single-point experiments performed to ensure that the eluate HMW was acceptable at the highest load HMW level and under worst-case operating conditions for the TMAE HiCap step (Sections 4.5 and 5.5).

4.4.3 Recovery

Analysis of the recovery data showed that this response was best described by a 2 factor-interaction (2FI) model (data not shown). After applying the backward selection with alpha=0.05 to identify the significant factors, the ANOVA table for the model identified B, C, AB, and AC as significant factors (Table 15). Additional details can be found elsewhere.

TABLE 15
ANOVA Table for Recovery

| | p-value | |
|---|---|---|
| Source | Prob > F | Comments |
| Model | <0.0001 | Significant |
| A-Loading | <0.0001 | |
| B-Load pH | <0.0001 | |
| C-Load Conductivity | <0.0001 | |
| AB | 0.0086 | |
| AC | 0.0040 | |
| Lack of Fit | 0.0516 | not significant |
| R-Squared | 0.9911 | |
| Adj R-Squared | 0.9874 | |
| Pred R-Squared | 0.9702 | |
| Adeq Precision | 65.993 | |

Overall, the model had high R² and adjusted R² values and the predicted R² value was in good agreement with the adjusted R² indicating that the model had good predictive ability. Although the lack of fit was not significant in the above model, the p-value was only slightly >0.05 i.e., there was a 5.16% probability that a lack of fit value this large could occur due to noise. Typically, a p-value>0.1 is desirable for insignificant lack of fit of the model. However, the relatively large lack of fit value might have been an artifact of the small pure error calculated from the replicates and not a true indication of any lack of fit between the experimental data and the model predictions. Diagnostic plots revealed that the distribution of residuals was normal, the variance was constant, and there was no influence by lurking variables on the response, which confirmed that the ANOVA was valid (data not shown). Analysis of the model plots (data not shown) showed that process recovery increased with decreasing loading, increasing load pH, and/or decreasing load conductivity.

4.4.4 Purity by Reduced and Non-Reduced LC90/GXII and Highest Single Impurity

A review of the results of the reduced and non-reduced LC90/GXII assays showed that the differences between the % Purity and % Highest Single Impurity (HIS) values for the DoE runs were within the range of variability of the respective assays. Therefore, the mean±3 standard deviations were determined to be the best predictor of these responses and no model was developed.

4.4.5 HCP

Analysis of HCP levels in the TMAE HiCap eluate pools showed that all values were very close to or below the limit of quantitation (LOQ) of the assay. Thus, no model was developed for this response.

4.4.6 DoE Study Summary

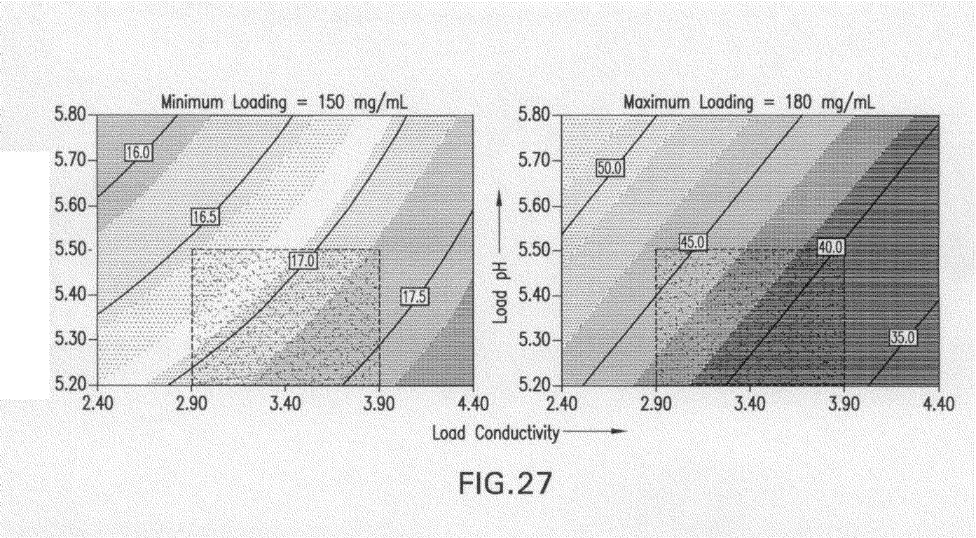

The predictive models developed for TSA and % Recovery were employed to identify suitable ranges for column loading and load pH and conductivity in order to achieve the desired level of TSA enhancement while maintaining acceptable product yield. Since lower SA enrichment is obtained at lower column loading, this condition represents the worst-case for product quality. Accordingly, as shown in the TSA model plot in FIG. 27, the minimum column loading for the TMAE HiCap step was set at 150 mg/mL in order to match or exceed the control sample TSA level of 16.0-16.5 mol/mol while providing reasonable ranges for load pH (5.2-5.5) and conductivity (2.9-3.9 mS/cm) for process robustness and good Manufacturing fit.

On the other hand, as aforementioned (Section 5.4.3), process recovery decreases with increasing column loading. Thus, there was a need to specify an upper range for column loading in Manufacturing to maintain reasonable process yields. Based on Manufacturing column sizing considerations as well as accounting for variability in the mass of starting material (i.e., ExcR-Fc biological product) and step yield of the upstream UF/DF step, a maximum loading of 180 mg/mL was determined for TMAE HiCap. As shown in the contour plot generated from the % Recovery model at the maximum loading condition, a minimum step yield of ˜36% was predicted. Overall, step yields between 36-59% can be expected for the TMAE HiCap process within the specified operating ranges for column loading and load pH and conductivity. Note: The load pH range was subsequently changed to 5.1-5.5 in order to provide a symmetrical operating range for Manufacturing i.e., 5.3±0.2. Based on the prediction plots below, this change is not expected to adversely impact process performance.

4.5 Additional Process Characterization

Additional single-point experiments were performed on the TMAE HiCap step within the operating ranges determined from the DoE study in order to evaluate the effects of other input parameters (e.g., load concentration and flow velocity) that were not examined in the earlier DoE study as well as to further assess process robustness. Results of these experiments are summarized in Table 16.

TABLE 16
Results of Additional Process Characterization Experiments

| Sample/ | Load Conc. | | | TSA |
|---|---|---|---|---|
| Run # | (mg/mL) | Recovery (%) | HMW (%) | (mol/mol) |
| PS10 Load | | | 2.0 | 11.8 |
| PS16 Load | | | 1.5 | 13.2 |
| 1^{a} | 4.9 | 44.9 | 2.3 | 17.5 |
| 2^{a} | | 46.6 | 2.2 | 17.4 |
| 3^{a} | | 47.6 | 1.8 | 16.9 |
| 4^{b} | 3.0 | 59.0 | 2.0 | 16.5 |
| 5^{b} | 5.2 | 59.7 | 1.7 | 16.0 |
| 6^{b} | 6.9 | 57.6 | 1.6 | 16.2 |
| 7^{c} | 3.0 | 34.6 | 2.7 | 18.5 |
| 8^{c} | 5.3 | 34.3 | 2.4 | 18.1 |
| 9^{c} | 6.9 | 35.6 | 2.3 | 18.3 |
| 10^{c} | 4.4 | 44.1 | 3.3 | 16.2 |
| 11 | | 48.8 | 3.5 | 15.7 |
| 12^{b} | | 51.3 | 3.2 | 16.0 |
| 13 | | 51.7 | 3.0 | 15.8 |

^{a}Setpoint conditions for column loading, load pH and load conductivity
^{b}Worst-case column loading, load pH and load conductivity for TSA, best-case for Recovery and HMW
^{c}Worst-case column loading, load pH and load conductivity for Recovery and HMW, best-case for TSA

The DoE experiments discussed in Section 5.4 employed diafiltered biological product from PS14 as the load material for the TMAE HiCap column. In order to evaluate the robustness of the TMAE HiCap step in the context of variability in the load material, experiments (Runs 2, 5, 8) were performed using a different biological product lot (i.e., PS16). The results of these runs were used as test points for verifying the predictive ability of the DoE models. As shown in Table 17, the actual values for % Recovery and TSA were within the 99% prediction intervals of the corresponding models. Thus, these DoE models were successfully verified. However, the measured HMW values were higher than the predicted ranges. This was due to the higher HMW levels in the load material employed for the verification runs (1.5% HMW in PS16 load) compared to the material used for the DoE experiments (0.9% HMW in PS14 load). Overall, DoE models generated using one lot of load material were able to successfiffly predict TMAE HiCap process performance for a different lot of column load material. Thus, the robustness of the TMAE HiCap process against lot-to-lot variability in the load material can be inferred from these results. In addition, the adsorbent lot employed for these runs was shown to have comparable performance as the lot used for the process development. Accordingly, this adsorbent lot was deemed to be suitable for use in the 2011 2K GMP Manufacturing campaign.

TABLE 17
Verification of DoE Models

| | Recovery (%) | | HMW (%) | | TSA (mol/mol) | |
|---|---|---|---|---|---|---|
| Run # | Actual | Predicted^{a} | Actual | Predicted^{a} | Actual | Predicted^{a} |
| 2 | 46.6 | 42.3-53.2 | 2.2 | 1.1-1.5 | 17.4 | 16.8-17.7 |
| 5 | 59.7 | 56.1-66.4 | 1.7 | 0.9-1.3 | 16.0 | 16.0-16.9 |
| 8^{b} | 34.3 | 27.5-41.7 | 2.4 | 1.2-1.7 | 18.1 | 17.0-18.4 |

^{a}Represents the 99% prediction intervals obtained from the corresponding models using the input parameters for each run
^{b}The column loading for this run was outside the design space of the DoE study. Thus, the predicted values were obtained by extrapolation of the models.

ExcR-Fc biological product produced during the 2010 2K GMP Manufacturing campaign had lower TSA levels than that observed for representative prototype runs (including PS 14 and 16). Therefore, an experiment was performed with PS10 material having TSA levels comparable to that observed for the GMP batches in order to determine the product quality obtained under worst-case conditions for TSA enrichment (Run 12). Analysis of the eluate pool for Run 10 showed that the TSA level was 16.0 mol/mol which was comparable to the TSA level measured for the control sample control i.e., 16.0-16.5 mol/mol. Thus, the TMAE HiCap process was shown to be capable of enriching the SA content of ExcR-Fc biological product to match the control sample even under these worst-case conditions.

In addition, HMW levels in representative prototype and GMP biological product lots were ≦1.6%. In order to evaluate the highest HMW level that can be expected in the TMAE HiCap eluate, Run 12 employed PS 10 load that had 2.0% HMW and was performed under worst-case operating conditions for HMW. As shown in Table 16, the eluate HMW level for Run 10 was 3.3%, which was less than the action limit of >3.5% specified for biological product. The use of GMP material with ≦1.6% HMW is expected to provide adequate safety margin for eluate HMW levels. Accordingly, the HMW increase observed over the TMAE HiCap step was deemed to be acceptable and no additional process steps were required in order to further reduce HMW levels in biological product.

The flow velocity of the TMAE HiCap process was varied between 75-250 cm/hr to evaluate the effect on product quality (Runs 1-3 and 11-13). The results for these runs showed that HMW levels in the eluate dropped with increasing flow velocity, while the product yield and TSA levels were comparable within the limits of assay variability. The observed HMW increase was likely due to the shorter residence time available for the larger HMW species to bind onto the column with increasing flow velocity. Thus, it is advantageous to operate at higher load velocity to minimize the HMW levels in the TMAE HiCap column eluate. Accordingly a flow velocity range of 150-250 cm/hr for the column load step was recommended for Manufacturing.

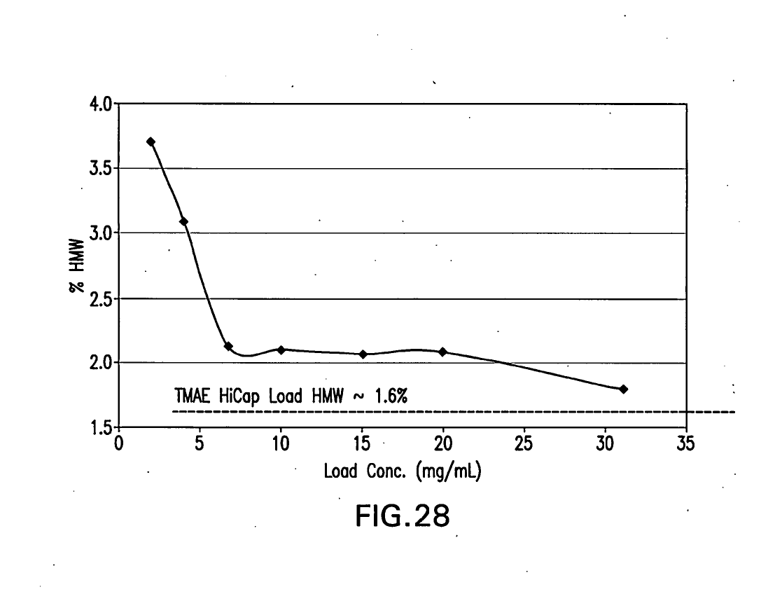

Runs 4-6 and 7-9 were performed to study the effect of load concentration on process performance. As shown in Table 16, HMW levels were observed to decrease as the load concentration increased from 3 mg/mL to 7 mg/mL. In addition, the decrease in eluate HMW levels was observed to be more significant as load concentration increased from 3 mg/mL to 5 mg/mL and compared to that observed for the increase in load concentration from 5 mg/mL to 7 mg/mL. At the same time, the process recovery and eluate TSA levels were comparable at the different load concentrations. Based on these results, additional single-point experiments were performed to determine the optimum load concentration to minimize the eluate HMW levels. As shown in FIG. 28, HMW levels initially increased as the load concentration increased to ˜7 mg/mL, which was consistent with previous results. However, a further increase in load concentration from 7-30 mg/mL provided no significant reduction in HMW levels. TSA analysis of these samples showed that comparable SA enrichment was obtained at the different load concentrations (data not shown).

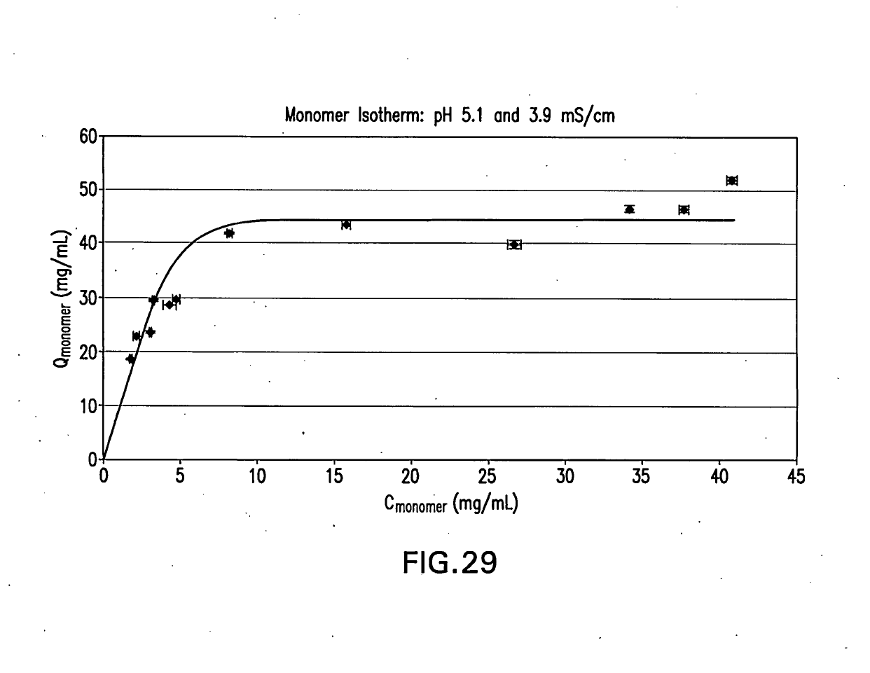

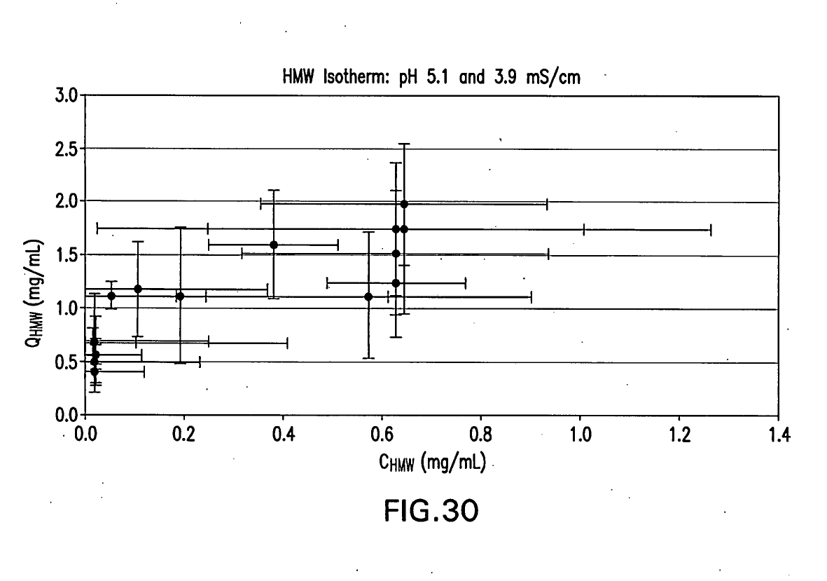

In order to further investigate this behavior, batch experiments were performed to measure the isotherms for monomer and HMW with the same load material that was used for the above experiments (9). The input load concentration for these experiments was varied between 2-45 mg/mL (Load HMW ˜1.6%), which represents the entire feasible operating range for the TMAE HiCap column. FIG. 29 shows the multicomponent adsorption isotherm for ExcR-Fc monomer on TMAE HiCap. As shown in the figure, the monomer concentration ranges from 0-˜5 mg/mL and ˜5-˜8 mg/mL represent the linear and non-linear regions of the isotherm, respectively. Within these regions, monomer binding increases with increasing concentration. The adsorbent is saturated at Cₘₒₙₒₘₑᵣ≧8 mg/mL and no additional monomer binding can occur. At the same time, the HMW isotherm showed very low levels of adsorption and there were no significant changes in HMW binding within the ranges of load concentration examined (FIG. 30). Taken together, these results demonstrate that between 0-8 mg/mL load concentration, Qₘₒₙₒₘₑᵣ on the column increased while Q_{HMW} did not change significantly. This resulted in the observed decrease in eluate HMW levels as the load concentration was increased to ˜8 mg/mL. Since monomer saturation was achieved at load concentrations≧8 mg/mL and Q_{HMW} remained largely unchanged, no further reduction in eluate HMW levels was obtained with increasing load concentration. Based on these results, a load concentration of 10±2 mg/mL was recommended for Manufacturing to minimize HMW levels in the TMAE HiCap column eluate.

### 5 CONCLUSIONS

A Phase I TMAE HiCap chromatography process was successfully developed for the enrichment of sialic acid (SA) levels in ExcR-Fc biological product. Initially, experiments were performed to evaluate the ability of different adsorbents and modes of chromatography to separate various ExcR-Fc isoforms on the basis of the differences in the degree of sialylation. To this end, cation exchange (SE HiCap), anion exchange (TMAE HiCap and Capto DEAE), hydrophobic interaction (Phenyl Sepharose), and hydroxyapatite (cHT Type I) chromatography were evaluated by linear gradient or step elution experiments. TMAE HiCap was selected for further development since it provided the highest degree of SA enhancement with the potential to match and even exceed the total sialic acid (TSA) levels of the control sample lots.

Subsequently, a novel “overloaded” bind and elute process was developed wherein the higher sialylated glycoforms having higher net negative charge and binding affinity to TMAE HiCap competed effectively for binding sites with the lower affinity lesser sialylated and non-sialylated glycoforms, thus displacing these lower affinity species. This resulted in the build-up of higher sialylated ExcR-Fc molecules on the adsorbent surface with increasing column loading. Subsequently, the column was eluted to recover the enriched higher sialylated glycoforms in the product pool, A DoE study was performed to optimize operating ranges for the key process parameters i.e., loading, load pH, and load conductivity. The results of these studies showed that the TSA levels in the TMAE HiCap eluate increased with increasing loading, decreasing load pH, and/or increasing load conductivity within the design space examined. On the other hand, product yield and eluate HMW levels showed the reverse trends within the same operating space. Accordingly, operating ranges were established for column loading and load pH and conductivity to ensure that product quality targets were achieved while maintaining reasonable process yields. In addition, single point experiments were also performed on TMAE HiCap to optimize the load concentration and flow velocity to improve product quality (specifically HMW levels) in the TMAE HiCap eluate.

Finally, single point experiments were performed to test the robustness of the TMAE HiCap process under worst-case operating conditions. These runs employed pilot-scale material having low TSA and high HMW levels (i.e., worst-case load PQ) to test the overall process capability. The results of these experiments clearly demonstrated that the TMAE HiCap process performed consistently, delivering product that met all pre-determined quality targets, even under worst-case operating conditions.

### 6 TMAE HICAP CHROMATOGRAPHY PROCESS DESCRIPTION

1) The column is equilibrated using ≧3 CV of 50 mM Tris+3M NaCl, pH 8.0 at ≦75 cm/hr in downflow.
2) The column is equilibrated using ≧5 CV of 55 mM acetate, pH 5.3 at ≧150 cm/hr in downflow.
3) The column is loaded to between 150-180 mg/mL at 150-250 cm/hr with load pool in downflow. [Note: Load pool pH=5.3±0.2 and conductivity=3.4±0.5 mS/cm].
4) The column is washed with 3 CV of 55 mM acetate, pH 5.3 at 150-250 cm/hr in downflow.
5) The column is eluted using 50 mM acetate+300 mM NaCl, pH 5.3 at ≦75 cm/hr in downflow. Elution collection is started immediately at the start of the elution block and the elution pool is collected 8 CV thereafter.
6) The column is stripped with ≧3 CV of 50 mM Tris+3M NaCl, pH 8.0 at ≦150 cm/hr in downflow.
7) The column is cleaned with ≧3 CV of 0.5 N NaOH at ≦150 cm/hr in downflow.
8) The column is cleaned with ≧3 CV of 50 mM Acetate+1M NaCl, pH 2.5 at ≦150 cm/hr in downflow.
9) The column is stored with ≧3 CV of 1% Benzyl alcohol+0.5M Acetic acid+16 mM NaOH, pH 3.2 at ≦150 cm/hr in downflow.

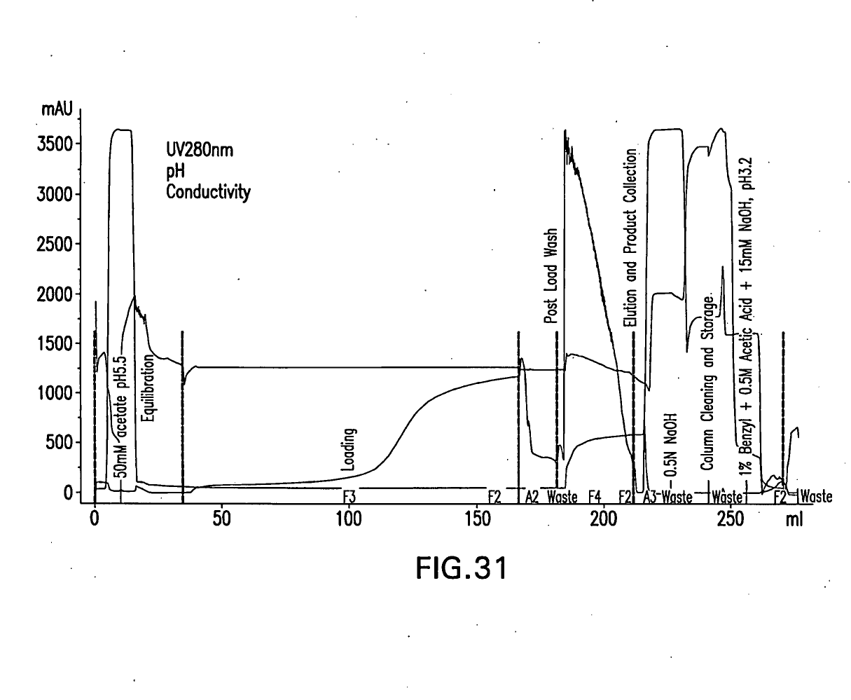

FIG. 31 shows a representative chromatogram for the overloaded bind and elute process developed for ExcR-Fc on TMAE HiCap. The different steps in the chromatography process are clearly outlined in this chromatogram.

1. A method for enhancing or increasing the concentration of biological product in a final mixture, wherein said biological product has one or more selected characteristics, wherein said method comprises:
(a) allowing an initial mixture of biological products with and without said selected characteristics to contact a chromatography medium wherein the quantity of biological products in said initial mixture exceeds the binding capacity or the dynamic binding capacity of said chromatography medium;
(b) allowing biological product not having said one or more selected characteristics to be separated by said chromatography medium; and
(c) recovering a final mixture of biological products from said chromatography medium wherein said final mixture comprises an enhanced or increased concentration of biological product with one or more selected characteristics, compared to the concentration of biological product in said initial mixture.

2. The method of claim 1, wherein said chromatography medium is selected from the group consisting of:
a) ion exchange medium;
b) anion exchange medium;
c) cation exchange medium;
d) hydroxyapatite medium;
e) hydrophobic interaction chromatography medium;
f) antibody-affinity medium;
g) immunoglobulin Fc-region affinity medium;
h) ligand-affinity medium;
i) receptor-affinity medium;
j) mixed-mode medium;
k) use of any two or more of a) through j) performed sequentially in any order.

3. The method of claim 1, wherein the binding capacity or dynamic binding capacity of said chromatography medium is exceeded by an amount selected from the group consisting of:
a) 10% or more;
b) 20% or more;
c) 30% or more;
d) 40% or more;
e) 50% or more;
f) 100% or more;
g) 200% or more;
h) 500% or more; and
i) 1000% or more.

4. The method of claim 1, wherein the binding capacity or dynamic binding capacity of said chromatography medium is exceeded by an amount selected from the group consisting of:
a) 1.5-fold or more;
b) 2-fold or more;
c) 3-fold or more:
d) 4-fold or more;
e) 5-fold or more;
f) 6-fold or more;
g) 7-fold or more;
h) 8-fold or more;
i) 9-fold or more;
j) 10-fold or more;
k) 20-fold or more;
l) 30-fold or more;
m) 40-fold or more;
n) 50-fold or more;
o) 100-fold or more; and
p) 500-fold or more.

5. The method of claim 1, wherein the amount of biological products recovered in the final mixture, compared to the amount of biological products in the initial mixture, is an amount selected from the group consisting of:
a) about 10% to about 80% recovered;
b) about 20% to about 60% recovered;
c) about 30% to about 60% recovered;
d) about 30% to about 50% recovered;
e) about 35% to about 50% recovered;
f) about 35% to about 45% recovered;
g) about 40% to about 45% recovered;
h) about 40% to about 50% recovered;
i) about 45% to about 50% recovered;
j) about 10% recovered;
k) about 15% recovered;
l) about 20% recovered;
m) about 25% recovered;
n) about 30% recovered;
o) about 35% recovered;
p) about 40% recovered;
q) about 45% recovered;
r) about 50% recovered;
s) about 55% recovered;
t) about 60% recovered;
u) about 65% recovered;
v) about 70% recovered;
w) about 75% recovered; and
x) about 80% recovered.

6. The method of claim 1, wherein the concentration of biological product with one or more selected characteristics is increased or enhanced, compared to the initial mixture of biological products, by an amount selected from the group consisting of:
a) at least about 5%;
b) at least about 10%;
c) at least about 20%;
d) at least about 30%;
e) at least about 40%;
f) at least about 50%;
g) at least about 60%;
h) at least about 70%;
i) at least about 80%; and
j) at least about 90%.

7. The method of claim 1, wherein the selected characteristic (or characteristics) is (are) selected from the group consisting of:
a) degree of net negative charge at a set pH value;
b) degree of net positive charge at a set pH value;
c) degree of hydrophobicity;
d) degree of hydrophilicity;
e) quantity and/or type of carbohydrate content;
f) quantity and/or type of N-linked glycosylation content;
g) quantity and/or type of O-linked glycosylation content;
h) total sialic acid content; and
i) any one or more of a) through h).

8. The method of claim 1, wherein the biological product is selected from the group consisting of:
a) a protein;
b) an antibody or fragment thereof;
c) a polypeptide comprising an extracellular receptor ligand-binding domain;
d) a receptor ligand;
e) a heterologous fusion protein;
f) a fusion protein comprising an immunoglobulin Fc-region; and
g) a fusion protein comprising an extracellular receptor ligand-binding domain and an immunoglobulin Fc-region.

9. The method of claim 1, wherein said method further comprises recovery of said biological product at a manufacturing scale.

10. The method of claim 8, wherein said method further comprises recovery of a therapeutically useful biological product.

11. The method of claim 2, wherein said method further comprises recovery of said biological product at a manufacturing scale.

12. The method of claim 3, wherein said method further comprises recovery of said biological product at a manufacturing scale.

13. The method of claim 4, wherein said method further comprises recovery of said biological product at a manufacturing scale.

14. The method of claim 5, wherein said method further comprises recovery of said biological product at a manufacturing scale.

15. The method of claim 6, wherein said method further comprises recovery of said biological product at a manufacturing scale.

16. The method of claim 7, wherein said method further comprises recovery of said biological product at a manufacturing scale.

17. The method of claim 8, wherein said method further comprises recovery of said biological product at a manufacturing scale.
